(19) United States
(12) Patent Application Publication
(10) Pub. No.: US 2012/0252774 A1
(43) Pub. Date: Oct. 4, 2012

(54) STEROID TETROL SOLID STATE FORMS - 2

(76) Inventors: Steven K. White, San Diego, CA (US); Erin E. Jansen, San Diego, CA (US)

(21) Appl. No.: 13/328,760

(22) Filed: Dec. 16, 2011

Related U.S. Application Data
(60) Provisional application No. 61/424,173, filed on Dec. 17, 2010.

(51) Int. Cl. C07J 1/00; A61P 29/00; A61K 31/565

(52) U.S. Cl. 514/182; 552/615

(57) ABSTRACT

The invention relates to solid state forms of androst-5-ene-3α,7β,16β,17β-tetrol, formulations containing or prepared from such solid state forms and use of these materials for modulating unwanted inflammation including acute and chronic non-productive inflammation. The formulations can be used to prevent, treat or slow the progression of conditions related to autoimmunity and metabolic disorders such as arthritis, multiple sclerosis, ulcerative colitis, Type 1 diabetes and Type 2 diabetes.

### CROSS REFERENCE TO RELATED APPLICATIONS

This nonprovisional U.S. patent application claims priority under 35 USC §119(e) from pending U.S. provisional application No. 61/424,173, filed on Dec. 17, 2010, which is incorporated herein by reference in its entirety.

### FIELD OF THE INVENTION

The field of the invention relates to solid state forms, including amorphous and crystalline forms, of androst-5-ene-3α,7β,16α,17β-tetrol and methods for their preparation. The invention further relates to solid formulations comprising one or more crystalline forms of androst-5-ene-3α,7β,16α,17β-tetrol and to methods for using the crystalline forms in preparing solid and liquid formulations and uses of these formulations for the treatment of inflammation-based or inflammation-driven diseases or conditions including autoimmune diseases, lung inflammation conditions, inflammatory bowel diseases, metabolic and cardiovascular conditions, neurodegenerative diseases and hyperproliferation conditions. Unit dosage forms for the solid and liquid formulations are also included.

### BACKGROUND OF THE INVENTION

The ability of a substance to exist in more than one crystalline form is generally referred to as polymorphism and these different crystalline forms are typically named “polymorphs” and may be referred to by certain analytical properties such their X-ray powder diffraction (XRPD) patterns. In general, polymorphism reflects the ability of a molecule to change its conformation or to form different intermolecular and intramolecular interactions. This can result in different atom arrangements that are reflected in the crystal lattices of different polymorphs. However, polymorphism is not a universal feature of solids, since some molecules can exist in one or more crystal forms while other molecules do not. Therefore, the existence or extent of polymorphism for a given compound is unpredictable.

The different polymorphs of a substance posses different crystal lattice energies and thus each such crystalline form typically shows one or more different physical properties in the solid state, such as density, melting point, color, stability, dissolution rate, flowability, compatibility with milling, granulation and compacting and/or uniformity of distribution [See, e.g., P. DiMartino, et al., J. Thermal Anal. 48:447-458 (1997)]. The capacity of any given compound to occur in one or more crystalline forms is unpredictable as are the physical properties of any single crystalline form. The physical properties of a polymorphic form may affect its suitability in pharmaceutical formulations. For example, those properties can affect positively or negatively the stability, dissolution and bioavailability of a solid-state formulation, which subsequently affects suitability or efficacy of such formulations in treating disease.

An individual crystalline form (i.e., a polymorphic form) having one or more desirable properties can be suitable for the development of a pharmaceutical formulation having desired property(ies). Existence of a compound with another specific crystalline form(s) that has an undesirable property(ies) can impede or prevent development of a desired polymorphic form of the compound as a pharmaceutical agent.

In the case of a chemical substance that exists in more than one polymorphic form, the less thermodynamically stable forms can occasionally convert to the more thermodynamically stable form at a given temperature after a sufficient period of time. When this transformation is rapid, such a thermodynamically unstable form is referred to as a “metastable” form. In some instances, such as in a suitable formulation, a metastable form may exhibit sufficient chemical and physical stability under normal storage conditions to permit its use in a commercial form.

### SUMMARY OF THE INVENTION

In one principal embodiment, the invention provides new amorphous and crystalline forms of 10R,13S-dimethyl 2,3,4,7,8R,9S,10,11,12,13,14S,15,16,17-hexadecahydro-1H-cyclopenta[a]phenanthrene-3S,7R,16R,17S-tetrol, which is represented by Formula 1A.

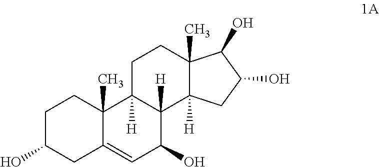

The Formula 1A compound (hereafter also referred to as androst-5-ene-3α,7β,16α,17β-tetrol or 3α-tetrol) has been prepared in various solid state forms and in particular amorphous and crystalline forms referred herein as Form Iα and Form IIα. Solid state forms of 3α-tetrol are suitable for treating acute or chronic conditions related to or associated with unwanted inflammation, including lung inflammation conditions, bowel inflammation conditions, liver inflammation conditions, metabolic and cardiovascular conditions, autoimmune conditions, hyperproliferation conditions, neurodegenerative conditions, ischemia-reperfusion injury and related conditions. Solid state forms of 3α-tetrol are also suitable for treating inflammation associated with ischemia-reperfusion or other injuries such as wounds.

Formulations comprising a solid state form of Compound 1A, wherein the solid state form is a crystalline form, include Form Iα or Form IIα substantially free or essentially free of amorphous 3α-tetrol, Form Iα substantially free or essentially free of Form IIα 3α-tetrol and Form IIα substantially free or essentially free of Form Iα 3α-tetrol. Other formulations comprising a solid state form of Compound 1A include amorphous 3α-tetrol, essentially free of 3α-tetrol in crystalline form.

Conditions related to metabolic conditions include hyperglycemia, insulin resistance and Type 2 diabetes (including forms with (1) predominant or profound insulin resistance, (2) predominant insulin deficiency and some insulin resistance and (3) forms intermediate between these). Conditions related to metabolic conditions also include obesity (usually patients having a body mass index of about 29, about 30 or more, or as diagnosed). Conditions related to metabolic conditions also include hyperlipidemia conditions such as hypertriglyceridemia and hypercholesterolemia.

In diabetes, the formulations described herein are useful to (1) enhance β-cell function in the islets of Langerhans (e.g., increase insulin secretion), (2) reduce the rate of islet cell damage, (3) increase insulin receptor levels or activity to increase cell sensitivity to insulin and/or (4) modulate glucocorticoid receptor activity to decrease insulin resistance in cells that are insulin resistant.

Conditions related to autoimmunity include Type 1 diabetes (including Immune-Mediated Diabetes Mellitus and Idiopathic Diabetes Mellitus), multiple sclerosis, optic neuritis, Crohn's disease (regional enteritis), ulcerative colitis, rheumatoid arthritis and Hashimotos' thyroiditis.

The solid state forms of Compound 1A described herein are thus useful to treat, prevent, ameliorate or slow the progression of conditions or their related symptoms related to or associated with unwanted inflammation that may be acute or chronic.

Formulations useful to treat, prevent, ameliorate or slow the progression of an inflammation, metabolic or autoimmune condition or a symptom associated thereto include formulations comprising one or more excipients and a crystalline hydrate of Compound 1A, including Form Iα 3α-tetrol, substantially free or essentially free of 3α-tetrol in anhydrate or amorphous form, a crystalline anhydrate of Compound 1A, including Form IIα 3α-tetrol substantially free or essentially free of 3α-tetrol in hydrate or amorphous form or amorphous Compound 1A substantially free or essentially free of 3α-tetrol in crystalline form.

One embodiment of the invention is directed to a particular crystalline form of 3α-tetrol (e.g., crystalline Form Iα or Form IIα) substantially free or essentially free of other solid state forms of 3α-tetrol.

Another embodiment of the invention is directed to 3α-tetrol in amorphous form substantially free or essentially free of other solid state forms of 3α-tetrol

Additional embodiments of the invention are directed to a particular crystalline hydrate or anhydrate of 3α-tetrol (e.g., Form Iα or Form IIα, respectively) or a mixture of a crystalline hydrate and a crystalline anhydrate of 3α-tetrol substantially free or essentially free of other solid state forms of 3α-tetrol

Other embodiments of the invention are directed to methods of preparation of a particular crystalline form of 3α-tetrol or 3α-tetrol in amorphous form.

In some embodiments a solid state form of Compound 1A is characterized or identified by methods comprising X-ray Powder Diffraction (XRPD) and one or more thermal methods including Differential Thermal Analysis (DTA), Differential Scanning Calorimetry (DSC), Modulated Differential Scanning Calorimetry (mDSC), Thermogravimetric Analysis (TGA), Thermogravimetric-infrared (TG-IR) analysis and melting point measurements.

In some embodiments a solid state form of Compound 1A is characterized or identified by methods including XRPD and a vibrational spectroscopy method such as Raman spectroscopy.

Other embodiments of the invention are directed to pharmaceutically acceptable formulations in solid form comprising a particular crystalline form of 3α-tetrol disclosed herein that is substantially free of other crystalline forms of 3α-tetrol and methods for preparation of the formulations.

Still other embodiments of the invention are directed to liquid formulations or invention compositions prepared by contacting or admixing at least one crystalline form of 3α-tetrol with a liquid excipient, optionally in the presence of another excipient, and methods for preparation of the liquid formulation.

Other embodiments that are related to contacting or admixing at least one solid state form of 3α-tetrol with a liquid excipient are directed to solid formulations as suspension formulation wherein at least some amount of 3α-tetrol is present as particles in the formulation. These suspension formulations are made using a solid state form described herein.

Yet another embodiment of the invention is directed to methods for treating a condition related to hyperglycemia and autoimmunity in a subject with a solid formulation comprising a solid state form of 3α-tetrol such as amorphous or a crystalline form of 3α-tetrol.

Another embodiment of the invention is directed to methods for treating a condition related to hyperglycemia and autoimmunity in a subject with a solid formulation comprising a solid state form of 3α-tetrol such as a crystalline form of 3α-tetrol.

Other embodiments of the invention are directed to uses of 3α-tetrol in solid state form (e.g., Form Iα, Form IIα or amorphous 3α-tetrol or a mixture thereof) to prepare a medicament for treatment of unwanted inflammation in a subject.

Other invention embodiments include methods of treating a pathological condition or one or more symptoms of a pathological condition associated with acute or chronic, non-productive inflammation using 3α-tetrol in crystalline form or a formulation or invention composition comprising this crystalline form.

Thus, additional embodiments of the invention include methods of treating a number of clinical conditions or symptoms thereof that are associated with acute inflammation, chronic inflammation or tissue damage from such conditions, which may be acute or chronic, with crystalline forms of 3α-tetrol as described herein, or solid or liquid formulations derived therefrom.

Other embodiments of the invention include methods to slow the progression of a number of clinical conditions that are associated with acute inflammation, chronic inflammation or tissue damage from such conditions, which may be acute or chronic.

In one preferred embodiment a solid state form of 3α-tetrol is used to treat a metabolic condition or an autoimmune condition in a subject such as a human or other mammal.

In another preferred embodiment a solid state form of 3α-tetrol is used to treat Type 2 diabetes or ulcerative colitis or other metabolic or autoimmune condition.

Additional embodiments and advantages of the present invention are described further in the following detailed description. The claimed agents and methods are also useful to reduce one or more symptoms associated with the conditions described herein.

Additionally, the use of the agents and methods described herein can be combined with one or more conventional treatments for each of these disorders.

### BRIEF DESCRIPTION OF THE FIGURES

FIG. 1. X-Ray powder diffraction pattern of Crystalline Form Iα androst-5-ene-3α,7β,16α,17β-tetrol

FIG. 2. Differential thermal and thermal gravimetric traces of Crystalline Form Iα androst-5-ene-3α,7β,16α,17β-tetrol

FIG. 3. Differential thermal and thermal gravimetric traces of Crystalline Form IIα androst-5-ene-3α,7β,16α,17β-tetrol

FIG. 4. X-Ray powder diffraction pattern of amorphous androst-5-ene-3α,7β,16α,17β-tetrol

FIG. 5. Solid phase Raman Spectrum of amorphous androst-5-ene-3α,7β,16α,17β-tetrol

FIG. 6. Differential thermal and thermal gravimetric traces of amorphous androst-5-ene-3α,7β,16α,17β-tetrol

### DETAILED DESCRIPTION

### Definitions

As used herein or otherwise stated or implied by context, terms that are defined herein have the meanings that are specified. The descriptions of embodiments and examples that are described illustrate the invention and they are not intended to limit it in any way. Unless otherwise contraindicated or implied, e.g., by mutually exclusive elements or options, in the descriptions or throughout this specification, the terms “a” and “an” mean one or more and the term “or” means and/or.

Unless specified otherwise explicitly or by context, percentage amounts are expressed as % by weight (w/w). Thus, a solid-dosage formulation containing at least about 2% Compound 1A (i.e., 3α-tetrol) in a solid-dosage formulation or suspension containing at least about 2% w/w 3α-tetrol. A solid unit-dose 3α-tetrol formulation containing 0.1% water means 0.1% w/w water is associated with that solid-dosage formulation, excluding water of hydration of a crystalline hydrate that is used to prepare the solid-dosage formulation.

“About” and “approximately,” when used in connection with a numeric value or range of values which is provided to describe a particular solid form, e.g., a specific temperature or temperature range, such as, for example, that describing a melting, dehydration, desolvation or glass transition; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid state form. Specifically, the terms “about” and “approximately,” when used in this context, indicate that the numeric value or range of values may vary by 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or 0.01% of the recited value or range of values while still describing the particular composition or solid state form.

“Solid State” as used herein refers to a physical state of a compound or composition comprising the compound, such as androst-5-ene-3α,7β,16α,17β-triol (i.e., 3α-tetrol); wherein at least about 2-10% of the mass of the compound that is present exists as a solid. Typically, the majority of the mass of 3α-tetrol will be in solid state form. More typically, between at least about 80-90% of the mass of 3α-tetrol is in solid form. Solid state forms include crystalline, disordered crystalline, polycrystalline, microcrystalline, nanocrystalline, partially crystalline, amorphous and semisolid forms or mixtures thereof, optionally with non-solid or non-crystalline 3α-tetrol. Solid state forms of Compound 3α-tetrol further include polymorphs, pseudopolymorphs, hydrates, solvates, dehydrated hydrates and desolvated solvates and mixtures thereof, optionally with non-solid or non-crystalline 3α-tetrol. Thus, solid state forms of 3α-tetrol will include a single polymorph form of 3α-tetrol, a single pseudo-polymorph form of 3α-tetrol, a mixture of two or more, typically two or three, polymorph or pseudo-polymorph forms of 3α-tetrol or a combination of any one of these solid state forms, optionally with non-solid or non-crystalline 3α-tetrol, provided that at least about 2-10% of the mass of 3α-tetrol is in solid form.

The term “crystalline” and related terms used herein, when used to describe a substance, component or product, means that the substance, component or product is crystalline as determined by visual inspection or usually with a suitable method, typically an X-ray diffraction method such as X-ray powder diffraction [See, e.g., Remington's Pharmaceutical Sciences, 18^{th} ed., Mack Publishing, Easton Pa., p173 (1990); The United States Pharmacopeia, 23^{rd} ed., pp. 1843-1844 (1995)].

The term “crystalline forms” and related terms herein refers to the various crystalline modifications of a given substance, including, but not limited to, polymorphs, solvates, hydrates, mixed solvates, co-crystals and other molecular complexes. A crystalline form may also be a mixture various crystalline modifications of a given substance such as a combination of pseudopolymorph or polymorph forms, a combination of one or more polymorph forms with one or more pseudopolymorph or a combination of such forms with amorphous or non-solid state forms of the substance. Typical combinations are of two or more polymorph or pseudo polymorph forms, such a mixture of a polymorph form with a pseudopolymorph form or a mixture of a polymorph or pseudopolymorph form with amorphous material. Typically crystalline forms are typically distinguishable from each other by their XRPD patterns. Solid state forms having different crystal morphologies but essentially identical XRPD patterns are considered to be different crystalline forms, since different morphologies can exhibit different properties related to physical shape. Properties related to physical shape include dissolution rate, stability, hygroscopicity, mechanical properties such hardness, tensile strength, compatibility (tableting) and those related to handling, e.g., flow, filtering, blending and other physical or pharmaceutical properties as described herein for different polymorphs.

“Polymorph” as used herein refers to a defined crystalline form of androst-5-ene-3α,7β,16α,17β-tetrol (i.e., 3α-tetrol). Polymorphs typically differ in their physical properties due to the order of the molecules in the lattice of the polymorph. Thus, polymorphs may exhibit one or more differences in physical or pharmaceutical properties including hygroscopicity, solubility, intrinsic dissolution rate, solid state reaction rates (i.e., chemical stability of a pharmaceutical ingredient as the drug substance or drug product), crystalline stability (i.e. tendency to transition to a more thermodynamically stable crystalline form), surface free energy, interfacial tension, mechanical strength (e.g., hardness, brittleness, plastic deformation, docility, malleability, etc.), tensile strength, compactability (i.e., tableting) and processability (e.g., handling, flow, blending, etc.). Differences in physical and mechanical properties of polymorphic forms of a drug substance may also affect scale-up and transfer from laboratory procedures though pilot plant and then to full production.

Polymorphs existing as hydrates, solvates or mixed solvates are generally referred to as pseudopolymorphs and represent different polymorphic or solid state forms in view of an isostructural polymorph form that is anhydrous or not a solvate. Pseudopolymorphs that differ in solvate identity or stoichiometry are also considered different polymorphic or solid state forms in view of each other. For example, 3α-tetrol existing as a solvate is a different solid state form in view of another solvate or an anhydrate (e.g., Form Iα or Form IIα). Stability profiles of hydrates and solvates at various temperatures and/or at different vapor pressures of water (e.g., relative humidity) or organic solvents will sometimes differ from those of the isostructural anhydrate or desolvate. Such differences may influence formulation, processing or stability of an active pharmaceutical ingredient (e.g., 3α-tetrol), either as the drug substance in a drug product under various storage conditions.

Thus, different crystalline or polymorphic forms may have different physical properties such as, for example, melting temperatures, heats of fusion, solubilities, and/or vibrational spectra as a result of the arrangement or conformation of the molecules in the crystal lattice (see, e.g., Byrn, S. R., Pfeiffer, R. R., and Stowell, J. G. (1999) Solid-State Chemistry of Drugs, 2^{nd} ed., SSCI, Inc.: West Lafayette, Ind.). The differences in physical properties exhibited by polymorphs and pseudopolymorphs may affect pharmaceutical parameters such as storage stability, compressibility and density (important in formulation and product manufacturing), and dissolution rate, which can be an important factor in bioavailability. Differences in stability may result from changes in chemical reactivity (e.g., differential oxidation, such that a dosage form discolors more rapidly when comprised of one polymorph or pseudopolymorph than when comprised of another polymorphic form) or mechanical changes (e.g., tablets crumble on storage as a kinetically favored polymorph converts to thermodynamically more stable polymorph) or both (e.g., tablets of one polymorph are more susceptible to breakdown at high humidity). As a result of kinetic solubility/dissolution rate differences, in the extreme case, some polymorphic transitions may result in lack of potency or, at the other extreme, toxicity. In addition, the physical properties of the crystal may be important in processing, e.g., one polymorph might be more likely to form solvates or hydrates that may be difficult to filter or wash free of impurities due to, for example, by differences in crystal morphology and/or particle size distribution.

Typically, crystalline forms are distinguished from each other by one or more physical or analytical properties such as rate of dissolution, Infrared and Raman spectroscopy, X-ray diffraction techniques such as single crystal and powder diffraction techniques, solid state-NMR(SS-NMR), thermal techniques such as melting point, differential thermal analysis (DTA), differential scanning calorimetry (DSC), thermal gravimetric analysis (TGA) and other methods as disclosed elsewhere in the specification. Additional methods to characterize or distinguish one pseudopolymorph from another polymorphic form, include elemental analysis, Karl-Fisher titration, dynamic vapor sorption analysis, thermogravimetric-infrared spectroscopic analysis (TG-IR), residual solvent gas chromatography and ¹H-NMR.

The term “isostructural crystalline form,” as used herein, refers to a crystal form of a substance that has a common structural similarity with another crystalline form, including approximately similar interplanar spacing in the crystal lattice. Thus, isostructural crystalline forms will have similar molecular packing motifs, but differing unit cell parameters (a symmetry translation). Due to their common structural similarity, isostructural crystalline forms typically have similar, but not necessarily identical, X-ray powder diffraction patterns. An isostructural crystalline form may be based upon a substance that is a neutral molecule or a molecular complex. The isostructural crystalline form may be a solvate, including a hydrate, or a desolvated solvate crystalline form of the substance. Isostructural forms that are solvates of a polymorph are sometimes referred to as pseudopolymorphic to the unsolvated polymorph. A solvated crystalline form typically contains one or more solvents, including water, in the crystal lattice, that may be the solvent or solvents of crystallization used in preparing the crystalline form.

“Amorphous”, as used herein, refers to a solid state form of a compound (e.g., 3α-tetrol) wherein in the three dimensional structure positions of the molecules relative to one another are essentially random, [for example, see Hancock et al. “Characteristics and significance of the amorphous state in pharmaceutical systems” J. Pharm. Sci Vol. 86, pp. 1-12 (1997)]. As a result, amorphous material will have only liquid-like short range order, and, when examined by X-ray diffraction, will generally produce broad, diffuse scattering that will result in peak intensity(ies) sometimes centered on one or more amorphous halos. Thus, XRPD analysis of amorphous material will provide a 2-theta pattern with one or more broad bands with no distinctive peaks.

Amorphous Compound 1A may sometimes be characterized by its glass transition temperature (T_{g}), which defines a pseudo second order phase transition in which a supercooled melt of 3α-tetrol yields, on cooling, a glassy structure with properties similar to those of crystalline 3α-tetrol. However, since T_{g} is a kinetic parameter, its value will be dependent on the melt cooling rate and the measurement conditions used for its determination (e.g., the slower the melt cooling rate, the lower T_{g} will be). Furthermore, T_{g} of an amorphous sample, such as amorphous 3α-tetrol will be highly dependent on the amount of water present. For example, a 1% increase in water content may lower T_{g} by about 10° C. or more. The glass transition temperature for a sample of amorphous 3α-tetrol may be obtained by differential scanning calorimetry (DSC), which will exhibit a heat capacity change having a second order endothermic transition that appears as a step transition. The inflection point of this transition provides T_{g}.

“Formulation” or “pharmaceutically acceptable formulation” as used herein refers to a composition comprising androst-5-ene-3α,7β,16α,17β-tetrol (i.e., 3α-tetrol), present in a solid state form, in addition to one or more pharmaceutically acceptable excipients or a composition prepared from 3α-tetrol and one or more pharmaceutically acceptable excipients. Formulations include compositions prepared from a solid state form of 3α-tetrol, wherein the composition is suitable for administration to a human. The formulation may be comprised of, or be prepared from amorphous 3α-terol or a mixture of a crystalline form of 3α-tetrol (i.e., Form Iα or Form IIα) and amorphous 3α-tetrol. Additionally, the formulation may be comprised of or prepared from a crystalline form of 3α-tetrol or be prepared from, one, two or more solid state forms of 3α-tetrol. Typically, formulations of 3α-tetrol will be comprised of or prepared from Form Iα or Form IIα, substantially free or essentially free of amorphous 3α-tetrol or amorphous 3α-tetrol substantially free or essentially free of 3α-tetrol in crystalline form. Preferred formulations of 3α-tetrol contain Form Iα or Form IIα predominately free or essentially free of other solid state forms of 3α-tetrol.

“Solid formulation” as used herein refers to a pharmaceutically acceptable formulation wherein 3α-tetrol is in solid state form in the presence of one or more pharmaceutically acceptable excipients wherein the majority of the mass amount of the solid state form of 3α-tetrol used in preparation of the formulation remains in that solid state form for at least about 6 months at ambient temperature, usually for at least about 12 months or 24 months at ambient temperature, when admixed with the excipients in proportions required for the solid state formulation. Dosage units that are a solid formulation include tablets, capsules, caplets, suspensions and other dosage units typically associated with oral administration of an active pharmaceutical ingredient in solid state form to a subject in need thereof.

“Liquid formulation” as used herein refers to a pharmaceutically acceptable formulation wherein one or more solid state forms of 3α-tetrol has been admixed or contacted with one or more pharmaceutically acceptable excipients, wherein at least one of the excipients is in liquid state form in proportions required for the liquid formulation, such that a majority of the mass amount of 3α-tetrol is dissolved into the non-solid excipient. Dosage units containing a liquid formulation include syrups, gels, ointments and other dosage units typically associated with parenteral or enteral administration of an active pharmaceutical ingredient to a subject in need thereof in non-solid state form.

“Suspension formulation” as used herein refers to a pharmaceutically acceptable formulation wherein one or more solid state forms of 3α-tetrol has been mixed or contacted with one or more pharmaceutically acceptable excipients, wherein at least one of the excipients is in liquid or non-solid state form (i.e. a non-solid excipient), in proportions wherein the majority of the mass amount of 3α-tetrol is not dissolved or is suspended in the non-solid state excipient or the excipient mixture of which the non-solid state excipient is comprised.

“Invention composition” as used herein refers to a mixture comprised of or prepared from one or more solid state forms of 3α-tetrol and one or more other components. Thus, an invention composition may be comprised of or prepared from one or more solid state forms of 3α-tetrol and one or more excipients and is a composition that may or may not be suitable for administration to a subject. In some embodiments, an invention composition consists essentially of pharmaceutically acceptable excipients and 3α-tetrol and may or may not require addition of another pharmaceutically acceptable excipient prior to administration to a subject by an intended route of delivery. For example, a lyophilized formulation containing or prepared from a solid state from of 3α-tetrol will typically require addition of a suitable liquid excipient prior to parenteral delivery by injection to a subject.

“Substantially free” as used herein refers to 3α-tetrol wherein more than about 60% by weight of the compound is present as the given solid state form. For example, the term crystalline 3α-tetrol “substantially free” of amorphous material refers to a solid-state form of 3α-tetrol wherein more than about 60% of 3α-tetrol is in one or more crystalline forms. Such compositions preferably contain at least about 80%, more preferably at least about 90%, of 3α-tetrol in one or more crystalline forms with the remaining present as non-crystalline 3α-tetrol. In another example, the term amorphous 3α-tetrol “substantially free” of crystalline 3α-tetrol refers to a solid-state form of 3α-tetrol wherein more than about 60% of 3α-tetrol is amorphous. Such compositions typically contain at least about 80%, preferably at least about 90%, more preferably at least about 95%, of amorphous 3α-tetrol, with the remaining present as crystalline 3α-tetrol. In yet another example, the term Form Iα “substantially free” of other crystalline forms refers to a solid-state composition of 3α-tetrol wherein more than about 60% of 3α-tetrol exists as Form Iα. Such compositions typically contain at least about 80%, preferably at least about 90%, more preferably at least about 95% 3α-tetrol as a single crystalline form. Preferred formulations of 3α-tetrol contain at least about 80%, preferably at least about 90% and more preferably at least about 95% of 3α-tetrol as Form Iα or Form IIα, with the remaining 3α-tetrol present as other solid state or non-solid state forms. Most preferred formulations contain about 95-99% of Form Iα or Form IIα 3α-tetrol with about 97%, about 98% or about 99% as a single crystalline form of 3α-tetrol particularly preferred.

“Essentially free” as used herein refers to a component so identified as not being present in an amount that is detectable under typical conditions used for its detection or would adversely affect the desired properties of a composition or formulation in which the component may be found. For example, “essentially free of liquid” means a composition or formulation in solid form that does not contain water or solvent, in liquid form, in an amount that would adversely affect the pharmaceutical acceptability of the formulation or composition for use in a solid dosage form to be administered to a subject in need thereof. A suspension is considered a solid formulation and for such formulations liquid excipient(s) comprising the suspension formulation are not included within this definition. “Crystalline Form Iα essentially free of amorphous 3α-tetrol” refers to a specific crystalline form of 3α-tetrol in which amorphous 3α-tetrol is not detected by XRPD analysis. Typically, the detection limit for amorphous material within crystalline material is about 10%.

“Substantially pure” as used herein refers to a solid state form of 3α-tetrol that contain less than about 3% or less than about 2% by weight total impurities, or more preferably less than about 1% by weight water, and/or less than about 0.5% by weight impurities such as decomposition or synthesis by-products or residual organic solvent. Residual solvent does not include solvent that is part of a crystalline solvate (i.e., a pseudopolymorph) such as water in a crystalline hydrate.

“Substantially identical” as used herein refers to measured physical characteristics that are comparable in value or data traces that are comparable in peak position and amplitude or intensity within the scope of variations that are typically associated with sample positioning or handling or the identity of the instrument employed to acquire the traces or physical characteristics or due to other variations or fluctuations normally encountered within or between laboratory environments or analytical instrumentation.

“Hydrate” as used here refers to a solid state form of 3α-tetrol that contains water molecules as an integral part of the solid state form and does not refer to water that is non-specifically bound to the bulk compound. Hydrates in a crystalline form can be isolated site hydrates or channel hydrates. Hydrates can contain stoichiometric or nonstoichiometric amounts of water molecules per compound molecule. Typically, water will be present in a crystalline hydrate in the ratio of 0.25, 0.5, 1.0, 1.5 or 2.0 relative to the compound of the crystalline hydrate on a mole basis.

“Solvate” as used here refers to a solid state form of 3α-tetrol that contains solvent molecules as an integral part of the solid state form and does not refer to solvent that is non-specifically bound to bulk compound. When the solvent molecule is water such solvates are sometimes referred herein as hydrates.

“Inflammation condition” as used herein refers to a condition that is characterized by the inappropriate or pathological presence of inflammation or its associated pain or fever. Inflammation may be acute or chronic and may be present in metastatic cancer, e.g., metastatic prostate or breast cancer, metabolic and cardiovascular conditions such as Type 2 diabetes and atherosclerosis, and autoimmune conditions such as ulcerative colitis. Acute inflammation may be present as a flare as for example in multiple sclerosis or arthritis.

Inflammation conditions include autoimmune conditions, such as multiple sclerosis, a lupus condition, e.g., systemic lupus erythematosus (an autoimmune condition), an arthritis condition, e.g., rheumatoid arthritis (an autoimmune condition), and an inflammatory bowel condition, e.g., ulcerative colitis or Crohn's disease (autoimmune conditions). Inflammation conditions also include metabolic conditions, such as hyperglycemia conditions, diabetes, liver inflammation conditions, e.g., nonalcoholic steatohepatitis (NASH), fatty liver conditions, acute and chronic lung inflammation conditions, e.g., obstructive pulmonary disease (COPD), acute asthma, chronic asthma, emphysema, acute bronchitis, allergic bronchitis, chronic bronchitis and lung fibrosis. Inflammation conditions further include neuroinflammation in neurodegenerative conditions such as Alzheimer's disease, Parkinson's disease, amyotrophic lateral sclerosis and age-related macular degeneration.

“Metabolic condition” as used herein include type 1 diabetes (an autoimmune condition), type 2 diabetes, obesity, metabolic syndrome, insulin resistance, hyperglycemia, impaired glucose utilization or tolerance, impaired or reduced insulin synthesis, a hyperlipidemia condition, such as hyperlipidemia, hypercholesterolemia, hypertriglyceridemia, elevated free fatty acids, or macrovascular damage, such as arterial atherosclerosis, hypolipidemias or vascular atherosclerosis. Hypercholesterolemia includes hyper-LDL cholesterolemia or elevated LDL cholesterol. Hypolipidemias include hypo-HDL cholesterolemia or low HDL cholesterol levels. Type 1 diabetes includes Immune-Mediated Diabetes Mellitus and Idiopathic Diabetes Mellitus. Type 2 diabetes includes forms with predominant or profound insulin resistance, predominant insulin deficiency and some insulin resistance and forms intermediate between these.

An “excipient”, “carrier”, “pharmaceutically acceptable carrier” or similar terms mean one or more component(s) or ingredient(s) that is acceptable in the sense of being compatible with the other ingredients in formulations or invention compositions comprising 3α-tetrol as the active pharmaceutical ingredient that is in solid state form when admixed with one or more of the excipients. These excipients usually are not overly deleterious to a subject to whom the composition formulation is to be administered. Excipients include one or more components typically used in the pharmaceutical formulation arts, e.g., one, two or more of fillers, binders, disintegrants, dispersants, preservatives, glidants, surfactants and lubricants. Exemplary excipients include povidone, crospovidone, corn starch, carboxymethyl cellulose, hydroxypropyl methylcellulose, microcrystalline cellulose, gum arabic, polysorbate 80, butylparaben, propylparaben, methylparaben, BHA, EDTA, sodium lauryl sulfate, sodium chloride, potassium chloride, titanium dioxide, magnesium stearate, castor oil, olive oil, vegetable oil, buffering agents such as sodium hydroxide, monobasic sodium phosphate, dibasic sodium phosphate, potassium hydroxide, monobasic potassium phosphate, dibasic potassium phosphate, tribasic potassium phosphate, potassium carbonate, potassium bicarbonate, ammonium hydroxide, ammonium chloride, saccharides such as mannitol, glucose, fructose, sucrose or lactose.

A “subject” means a human or an animal. Usually the animal is a mammal or vertebrate such as a non-human primate, dog or rodent.

A “surface-active agent” (surfactant) means a substance, which, at low concentrations, interacts between the surfaces of a solid and fluid in which the solid is insoluble or sparingly soluble. The fluid may be a liquid excipient present in a suspension formulation that comprises a solid state form of an active pharmaceutical ingredient, such as a solid state form of 3α-tetrol, the liquid excipient and a surface active agent that acts to improve suspendability.

Alternatively, the surface active agent may be present in an oral solid dosage form comprising the active pharmaceutical ingredient as a polymorphic form of 3α-tetrol (e.g., crystalline Form Iα or Form IIα or a mixture thereof) and the surface active agent, which acts to improve dissolution rate of the active pharmaceutical ingredient in gastric fluid. Surface-active agents are amphipathic in structure having both polar (hydrophilic) and non-polar (hydrophobic) regions in the same molecule. Examples of surface active agents used in the formulation arts are given in Corrigan, O. I.; Healy, A. M. “Surfactants in Pharmaceutical Products and Systems” in Encyclopedia of Pharmaceutical Technology 2^{nd} ed. Taylor and Francis, 2006, pp. 3583-3596.

A “suspension” generally refers to a solid state form of 3α-tetrol that is present, usually as a finely divided (e.g., micronized) crystalline solid, in a liquid carrier (vehicle) at a time prior to administration of the suspension. The suspension may be either ready to use or a dry powder reconstituted as a suspension dosage form just prior to use. Suspensions typically include a suspending or flocculating agent, a wetting agent, if the suspending or flocculating agent that is present does not already serve this purpose In a colloidal suspension, the 3α-tetrol particles are typically less than about 1 μm in size. In a coarse suspension, they are larger than about 1 μm. The practical upper limit for individual suspendable particles in coarse suspensions is about 50 μm to 75 μm although some proportion of particles up to 200 μm may be suitable dependent upon the syringeability of the suspension. Design considerations for developing a suspension for oral or parenteral administration are given in Akers, et al. J. Parenteral Sci. Tech. 1987 Vol. 41, pp. 88-96; Nash, R A “Suspensions” in Encyclopedia of Pharmaceutical Technology 2^{nd} ed. Taylor and Francis, 2006, pp 3597-3610 (which is hereby incorporated by reference herein).

Formulations.

In some embodiments a formulation comprising or prepared from one or more solid state forms of 3α-tetrol is administered parenterally to a subject having or subject to developing a disease or condition associated with acute or chronic non-productive inflammation. Invention compositions or formulation suitable for use in parenteral administration for human or veterinary applications include liquid solutions, suspensions, emulsions, gels, creams, intramammary infusions, intravaginal delivery systems and implants. Formulations or unit dosage forms suitable for use in oral administration include capsules, caplets, sachets, gelcaps and tablets.

Invention compositions and formulations will comprise or be prepared from androst-5-ene-3α,7β,16α,17β-tetrol (i.e., 3α-tetrol) in solid state form and one or more excipients The excipients are components or ingredients of an invention composition or formulation other than other than the active pharmaceutical ingredient (i.e., 3α-tetrol) that has been found acceptable in the sense of being compatible with the other ingredients or components and has been appropriately evaluated for safety and found not overly deleterious to the patient or animal to which the tetrol compound is to be administered. Compatibility and safety criteria to qualify as an excipient does not mean the excipient is devoid of any chemical, biological or pharmacological activity nor does it require complete inertness. Rather, whatever chemical, biological or pharmacological activities an excipient does posses, the level of activities are acceptable in view of the disease or condition being treated.

Formulations and invention compositions for parenteral administration of 3α-tetrol will usually employ a vehicle as a liquid diluent that provides, e.g., a liquid solution for intravenous injection (i.v.) or a liquid solution or suspension for introduction of 3α-tetrol by intramuscular (i.m.), intradermal or subcutaneous (s.c.) injection. The vehicle may be an oil which forms a solution, suspension or emulsion, that is suitable for non-intravenous routes of parenteral administration, or which form a solution, suspension, emulsion, gel or cream that is suitable for non-injection dependent routes of parenteral administration. A dry powder may be packaged with a propellant to permit nasal or pulmonary deliver of 3α-tetrol, usually as a micronized powder.

Formulations and invention compositions of the present invention may also include tonicity-adjusting agents, particularly in injectable parenteral formulations containing or prepared from one or more crystalline forms of 3α-tetrol. Suitable tonicity adjusting agents are for instance sodium chloride, sodium sulfate, dextrose, mannitol and glycerol, typically mannitol or dextrose.

Buffers agents can include for example those derived from acetic, aconitic, citric, glutaric, lactic, maelic, succinic, phosphate and carbonic acids, as known in the art. Example of buffering agents commonly used in parenteral formulations and of their usual concentrations can be found in Pharmaceutical Dosage Form: Parenteral Medications, Volume 1, 2^{nd} Edition, Chapter 5, p. 194, De Luca and Boylan, “Formulation of Small Volume Parenterals” at Table 5 (Commonly used additives in Parenteral Products). Sometimes the buffering agent is phosphate or citrate buffer present in a buffering agent range between about 10-100 mM to provide a suspension or solution at an initial pH in a pH range between about 4-9, typically between about 4-8.

For parenteral dosage forms, an anti-microbial preservative can be used if no other excipient serves this purpose. Suitable preservatives include, e.g., phenol, resorcinol, chlorobutanol, benzylalcohol, alkyl esters of para-hydroxybenzoic acid such as methyl, ethyl, propyl, butyl and hexyl (generically referred to as parabens), benzalkonium chloride and cetylpyridinium chloride. Sometimes the preservative is an edetate such as a pharmaceutically acceptable salt of EDTA, which may also serves as a metal chelator. Typically, an anti-microbial preservative is present in a preservative range between about 0.001% to 1.0% w/v, typically between about 0.1 to 0.4% or more typically about 0.02%.

Unless otherwise stated or implied by context, expressions of percentage of a liquid excipient in a formulation or an invention composition is given by v/v. Thus, 20% PEG 300 means 20% v/v PEG 300 is present in a liquid or suspension formulation or invention composition. Furthermore, a ratio expression of an amount of a solid excipient in a liquid or suspension formulation or invention composition refers to the excipient's weight relative to the weight of 3α-tetrol present in the liquid or suspension to total volume. Excipients in formulations for use in administration to humans may be NF or USP grade.

In preparing any of the formulations or invention compositions or that are disclosed herein and that comprise or are prepared from one or more crystalline forms of 3α-tetrol (and optionally one or more excipients), one may optionally mill, sieve or otherwise granulate crystalline 3α-tetrol or a formulation or invention composition or comprising crystalline particles of 3α-tetrol in order to obtain a desired average particle size.

Milling may occur before or after the crystalline particles of 3α-tetrol are contacted with one or more excipients. For example, one may mill a crystalline form of 3α-tetrol to obtain a mean particle diameter or a (Dv, 0.90) mean volume diameter of about 0.05-200 microns or about 0.5-30 microns (e.g., about 5, about 10, about 15, about 20, about 25, about 30, about 40, about 60, about 80, about 100 or about 120 microns mean volume weighted particle size or average diameter) before contacting the milled 3α-tetrol particles with a liquid or solid excipient(s).

Micronization may be accomplished by mechanical milling, ultrasonic disintegration, microfluidization, melt extrusion, spray drying, spray freeze-drying or precipitation. Micronization techniques are described in Drug Delivery Technology 2006, 6:54-60; Serajuddin, A T M J. Pharm. Sci. 1999, 88:1058-1066 (hereby specifically incorporated by reference into the present application). Micronization methods using mechanical milling include milling by ball mills, pin mills, jet mills (e.g., fluid energy jet mills). Other methods for micronization include grinding, sieving and precipitation of a compound(s) from a solution, (see, e.g., U.S. Pat. Nos. 4,919,341, 5,202,129, 5,271,944, 5,424,077 and 5,455,049, which are incorporated by reference herein). Particle size is determined by, e.g., transmission electron microscopy, scanning electron microscopy, light microscopy, X-ray diffractometry and light scattering methods or Coulter counter analysis (see, for example, “Characterization of Bulk Solids” D. McGlinchey, Ed., Blackwell Publishing, 2005) The 3α-tetrol crystals can be micronized separately or co-micronized with a surface-active agent, wetting agent or other carrier.

Since grinding, milling, micronization or other mechanical manipulations may result induce a change in polymorph form due to the energy imparted, the particles after such manipulations should be re-evaluated for polymorphism. Sometimes if the mechanical process is sufficiently intensive, the diminished long range ordering and increase presence of crystal defects may occur to an extent that crystalline 3α-tetrol appears present in non-crystalline rather than in a crystalline solid sate form when analyzed by standard XRPD analysis. For example, at average particle sizes lower than 50 angstroms (i.e., 0.005 μm), line widths in XRPD spectra will usually increase beyond 2 2θ (see Jenkins and Snyder “Introduction to X-ray powder diffractometry” Chemical Analysis Series Vol. 38, Wiley-Interscience, 1996). To more readily determine the crystalline form of 3α-tetrol by XRPD after micronization or other such mechanical manipulations, atomic pair distribution functions as described in WO 2005/082050 may be used.

Particle size, unless otherwise specified, refers to a number weighted mean diameter. Sometimes the particle size will be associated with a volume-weighted distribution known as a mean volume diameter and thus the particle size will be the diameter of particles, within a stated fraction (Dv) in a volume-weighted distribution of particles that will have the stated diameter. For example, a particle diameter represented by 35 μm (Dv, 0.90) means that 90% or more of the mass of particles will have a diameter of 35 μm or less. Particle size is determined by, e.g., transmission electron microscopy, scanning electron microscopy, light microscopy, X-ray diffractometry and light scattering methods or Coulter counter analysis (see, for example, “Characterization of Bulk Solids” D. McGlinchey, Ed., Blackwell Publishing, 2005).

For administration of an aqueous-based parenteral dosage form, a sterilized drug product is usually required. A solution dosage form may be sterilized by passage through a microbe-retaining filter or by heat sterilization whereas a suspension dosage from requires sterilization by input of energy. Alternatively, one or more crystalline forms of 3α-tetrol in a blend of solid excipients may be sterilized by ionizing radiation (“cold sterilization” method) and a sterile liquid diluent or a blend of excipients dissolved in the diluent is then added to the solids so sterilized under sterile conditions. Typically, conditions employed for cold sterilization reach 25-30 kGy. Sterilization procedures are discussed in FDA guidance to industry “Sterile drug products produced by aseptic processing” accessible at http://www.fda.gov/cber/gdlns/steraseptic.pdf.

Dosage Forms.

Typically, unit doses contain 0.5-500 mg, and more often about 1 mg to about 200 mg, of androst-5-ene-3α,7β,16α,17β-tetrol (i.e., 3α-tetrol). Unit dosage forms include those suitable for oral or parenteral dosing. Preferred unit dosage forms for oral dosing are tablets, capsules, caplets, gel caps and the like.

To limit the amount of water that reaches formulations or invention compositions containing humidity sensitive crystalline forms of 3α-tetrol (e.g., anhydrate crystalline forms including Form Iα or Form IIα) the formulations and invention compositions may be packaged in hermetically or induction sealed containers. Water permeation characteristics of such containers have been described, e.g., in Containers—Permeation, USP Chp. 23, 1787 et seq., United States Pharmacopeial Convention, Inc., 12601 Twinbrook Parkway, Rockville, Md. 20852, 1995.

Some embodiments of treating a subject using the invention composition or formulation described herein further include monitoring the subject's response to a particular dosing regimen or schedule, e.g., to any continuous or other administration method disclosed herein. For example, while dosing a subject who has an inflammation-based or inflammation-driven diseases or condition one can measure the subject's response, e.g., amelioration of one or more symptoms such as pain or fever or a change in a pro-inflammatory cytokine level. Once a response is observed dosing can be continued for one, two or three additional days, followed by discontinuing the dosing for at least one day (at least 24 hours). Once the subject's response shows signs of recurrence (e.g., a symptom begins to intensify or pro-inflammatory mediator level(s) begins to increase), dosing can be resumed for another course. An aspect of the subject's response to a formulation or invention composition containing 3α-tetrol is that the subject may show a measurable response within a short time, usually about 5-10 days, which allows straightforward tracking of the subject's response, e.g., by monitoring a symptom or a disease biomarker or expression of a pro-inflammatory cytokine or interleukin by e.g., white blood cells or a subset(s) thereof.

For any of the treatments or methods described herein, prolonged beneficial effects or a sustained anti-inflammatory response by a subject may result from a single administration or a few daily administrations of a formulation comprising or prepared from one or more crystalline forms of 3α-tetrol or from intermittent treatment with the 3α-tetrol formulation.

In some embodiments, invention compositions or formulations comprising or prepared from amorphous or one or more crystalline forms of 3α-tetrol may be used to treat, prevent or slow the progression of or ameliorate one or more conditions in a subject having or subject to developing a chronic, nonproductive inflammation condition wherein the inflammation condition is associated with a metabolic disorder or cardiovascular condition, e.g., hyperglycemia, diabetes or atherosclerosis.

In some embodiments, formulations or invention compositions comprising or prepared from amorphous or one or more crystalline forms of 3α-tetrol may be used to treat, prevent or slow the progression of or ameliorate one or more conditions in a subject having or subject to developing a inflammatory lung condition, e.g., asthma, acute respiratory distress syndrome, emphysema or COPD. In other embodiments, such formulations or compositions may be used to treat, prevent or slow the progression of or ameliorate one or more conditions in a subject having or subject to developing chronic bronchitis.

In some embodiments, formulations or invention compositions comprising or prepared from amorphous or one or more crystalline forms of 3α-tetrol may be used to treat, prevent or slow the progression of or ameliorate one or more conditions in a subject having or subject to developing a autoimmune disease, e.g., ulcerative colitis, Crohn's disease, multiple sclerosis or arthritis.

In some embodiments, formulations or invention compositions comprising or prepared from amorphous or one or more crystalline forms of 3α-tetrol may be used to treat, prevent or slow the progression of or ameliorate one or more conditions in a subject having or subject to developing a neurodegenerative disease associated with neuroinflammation, e.g., Alzheimer's disease, amyotrophic lateral sclerosis or Parkinson's disease.

Chronic, non-productive inflammation is the inappropriate presence of an unresolved inflammatory response that may become disconnected from any stimulus that may have been responsible for initiating the response. The unresolved inflammatory response may be present at an asymptomatic and chronically low level that worsens to a disease state or be symptomatic with occasional, unexpected intensification (e.g., a flare as in an autoimmune disease such as multiple sclerosis). Oftentimes, the unresolved inflammation transforms into a disease state that can be further perpetuated by the underlying inflammation (e.g. metabolic syndrome transitioning to type 2 diabetes). In some instances, a disease state is established irrespective of the initial presence of any inflammatory response, but is perpetuated by an inflammatory response produced by the disease state that becomes chronic (e.g. Alzheimer's disease). In other instances an established unresolved inflammatory response produced by or associated with a disease state sequel can sometimes progress into another disease state with more serious consequences (e.g., ulcerative colitis transitioning to colon cancer) or it may assist in worsening or propagating the disease state (e.g., promoting tumor metastasis in cancer).

The chronic or acute inflammatory-based diseases or conditions described herein to be treated with 3α-tetrol or solid or liquid formulations or invention compositions derived therefrom, may be mild and relatively newly diagnosed or more progressed and moderate to severe. In moderately to severely affected patients, 3α-tetrol will typically slow the progression of the condition or ameliorate one or more symptoms such as memory loss, dementia, fever, pain or insulin resistance. Such symptoms and treatment effects include, e.g., (a) reduced abdominal pain, bleeding or tissue damage associated with an inflammatory bowel disease, which may be associated with progression of the condition or intestinal tissue damage, (b) decreased hyperglycemia in type 2 diabetes patients, type 1 diabetes patients or obese or hyperglycemic pre-diabetic patients who may be prone to developing diabetes, (c) decreased mood swings, confusion, depression, agitation, short term memory impairment or insulin resistance in patients diagnosed with Alzheimer's disease or other neurological disorders and (d) reduced fatigue, weakness or liver tissue damage or reduced elevation of liver enzyme(s) (AST, SGOT, ALT, SGPT) or liver fibrosis in NASH or liver cirrhosis, which enzyme(s) elevation may be asymptomatic or not. Similar effects are also expected in patients having a symptom associated with acute inflammation in, for example, a bone fracture or a stroke, e.g., reduced pain or tissue damage.

Treatment of patients having one of the clinical conditions described herein will typically begin after the condition has been diagnosed, but the treatment can also be prophylactic and started when a patient is considered to be susceptible to developing a given condition, e.g., elderly patients having some age-associated memory loss or other cognitive impairment or patients having early stage Alzheimer's disease with limited dementia can be treated to slow the progression or delay the onset of the condition or to limit the severity of a symptom(s). Such treatment or prophylactic effects may be observed by comparison with untreated patients having a similar age, gender, medical history and/or disease profile or condition.

Conditions of unresolved chronic or acute inflammation to be treated with a crystalline form of androst-5-ene-3α,7β,16α,17β-tetrol (i.e., 3α-tetrol) or a formulation or invention composition comprising or derived from the crystalline from (i.e., solid or liquid formulations prepared using a crystalline form of 3α-tetrol), include metabolic conditions, autoimmune conditions, lung inflammation conditions, inflammatory bowel conditions, neurodegenerative conditions, hyperproliferation conditions, ischemic conditions, chemical or thermal burns and bone loss or bone damage conditions described herein.

Autoimmune conditions to be treated with a crystalline form of 3α-tetrol or a formulation derived therefrom include a lupus condition such as systemic lupus erythematosus and discoid lupus, rheumatoid arthritis, multiple sclerosis, myasthenia gravis, Graves disease, Sjögren's syndrome, Hashimotos thyroiditis, Crohn's disease and type 1 diabetes.

Lung inflammation conditions to be treated with a crystalline form of 3α-tetrol, or a formulation derived therefrom, include chronic obstructive pulmonary disease (COPD), acute asthma, chronic asthma, emphysema, acute bronchitis, allergic bronchitis, allergic respiratory disease, chronic bronchitis, pleurisy, allergic bronchopulmonary aspergillosis, chronic interstitial pneumonia, respiratory bronchiolitis-associated interstitial lung disease, cystic fibrosis, or fibrosing alveolitis (lung fibrosis) conditions such as subepithelial fibrosis in patients having chronic bronchitis.

Metabolic conditions to be treated with a crystalline form of 3α-tetrol, or a formulation derived therefrom, include metabolic syndrome, Type 2 diabetes, Type 1 diabetes and hyperglycemia. Other metabolic conditions to be treated are hyperlipidemia hypertriglyceridemia or hypercholesterolemia. Other metabolic conditions to be treated are liver cirrhosis conditions, nonalcoholic steatohepatitis (NASH) or nonalcoholic fatty liver disease (NAFLD).

Inflammatory bowel conditions to be treated with a crystalline form of 3α-tetrol, or a formulation derived therefrom, include ulcerative colitis, Crohn's disease or inflammatory bowel syndrome.

Neurodegenerative diseases to be treated, e.g., by slowing progression of the disease, using a crystalline form of 3α-tetrol, or a formulation derived therefrom, include Alzheimer's disease. Other neurodegenerative diseases to be treated are Parkinson's disease, dementias or a cognitive impairment condition without dementia, Huntington's disease and Amyotrophic lateral sclerosis (ALS).

Hyperproliferation or cancer conditions to be treated, e.g., by slowing progression of the disease, using a crystalline form of 3α-tetrol, or a formulation derived therefrom, include breast cancer, prostate cancer, small cell lung carcinoma, endometriosis and hyperplasia conditions such as benign prostatic hyperplasia.

Acute, non-productive inflammation conditions or tissue damage from these conditions to be treated with a crystalline form of 3α-tetrol, or a formulation derived therefrom, include skin lesions or disruptions, e.g., associated with wounds, keratosis or psoriasis, an ischemia condition, e.g., myocardial infarction, stroke and other central nervous system ischemia conditions such as brain hemorrhage, thromboembolism and brain trauma, bone loss or damage conditions, e.g., osteoarthritis and osteoporosis conditions such as postmenopausal osteoporosis, idiopathic osteoporosis or osteoporosis associated with treatment with a glucocorticoid, e.g., dexamethasone, prednisone, cortisone, corticosterone, etc.

A number of factors may contribute to the establishment and maintenance of some of the chronic inflammation conditions described herein with production of pro-inflammatory cytokines and chemokines being a common feature. For example, tumor necrosis factor-α (TNF-α) is a cytokine that is released primarily by mononuclear phagocytes in response to a number of immuno-stimulators. When administered to animals or humans, it causes inflammation, fever, cardiovascular effects, hemorrhage, coagulation, and acute phase responses similar to those seen during acute infections and shock states. Normal TNF-α levels are needed to elicit a number of normal immune responses. Excessive or unregulated TNF-α production may play a role in a number of disease conditions. These conditions include endotoxemia and/or toxic shock syndrome, e.g., Tracey et al., Nature 330:662-664 (1987) and Hinshaw et al., Circ. Shock 30: 279-292 (1990), cachexia, e.g., Dezube et al., Lancet, 335(8690): 662 (1990) and ARDS where high TNF-α concentrations have been detected in pulmonary aspirates from ARDS patients, e.g., Millar et al., Lancet 2(8665): 712-714 (1989). Excessive levels of TNF-α may also be involved in bone resorption diseases, including arthritis.

In chronic, non-productive inflammation, activated monocytes and neutrophils typically play a role in mediating inflammation associated pathology in some of the conditions or diseases attributable to this unresolved inflammatory state. Activated neutrophils can have increased production of pro-inflammatory cytokines. Neutrophils can be a source of toxic oxygen species whose generation mediates, at least in part, TNF-α secretion by activated macrophages. TNF-α may be necessary for some of the organ injury and failure that can be seen in sepsis.

Formulations that contain a solid state form of 3α-tetrol and formulations, e.g., solutions, that contain 3α-tetrol obtained from a solid state form of 3α-tetrol can be used to decrease excessive levels of one or more inflammation mediators such as TNF-α, IL-12, IL-23 or monocyte chemoattractant protein-1.

X-ray Powder Diffraction Analysis (XRPD)—

XRPD is typically used to characterize or identify crystal compositions (see, e.g., U.S. Pharmacopoeia, volume 23, 1995, method 941, p 1843-1845, U.S.P. Pharmacopeia Convention, Inc., Rockville, Md.; Stout et al, X-Ray Structure Determination; A Practical Guide, MacMillan Co., New York, N.Y. 1968). The diffraction pattern obtained from a crystalline compound is often diagnostic for a given crystal form, although weak or very weak diffraction peaks may not always appear in replicate diffraction patterns obtained from successive batches of crystals. This is particularly the case if other crystal forms are present in the sample in appreciable amounts, e.g., when a polymorph of a crystal has become partially hydrated, dehydrated, desolvated or heated to give a significant amount of another crystalline form.

The relative intensities of bands, particularly at low angle X-ray incidence values (low 2θ), may vary due to preferred orientation effects arising from differences in, e.g., crystal habit, particle size and other conditions of measurement. Individual XRPD peaks in different samples are generally located within about 0.3±1 2θ degree for broad peaks. Broad XRPD peaks may sometimes appear as two or more individual peaks located closely together. For sharp isolated peaks under reproducible conditions, the peak is usually found within about 0.2 2θ degrees on successive XRPD analyses. Thus, when a sharp isolated XRPD peak at a given position is identified as being located at, e.g., about 16.1, this means that the peak is at 16.1±0.1. It is usually not necessary to rely on all bands that one observes for a given crystalline form disclosed herein; sometimes even a single band may be diagnostic for a given polymorphic form of 3α-tetrol.

Typically, individual crystalline forms of 3α-tetrol are characterized by reference to 2, 3 or 4 XRPD peaks having the most intensity or the 2, 3 or 4 most reproducible peaks XRPD peaks and optionally by reference to one or two other physical or analytical properties such as melting point, one or more thermal transitions observed in DTA and/or differential scanning calorimetry (DSC), one or more absorption peaks observed in infrared spectroscopy (IR) and/or dissolution rate (DR) data in an aqueous or other solvent system. Standardized methods for obtaining XRPD, DTA, DSC, DR, etc. data have been described (see U.S. Pharmacopoeia, volume 23, 1995, United States Pharmacopeial Convention, Inc., Rockville, Md., pp 2292-2296 and 2359-2765).

Prominent XRPD peaks are preferably selected from observed peaks by identifying non-overlapping, low-angle peaks. A prominent peak will have relative intensity of at least about 5% or more typically at least about 10% or at least about 15% or at least about 20% relative intensity in comparison to the most intense peak in the X-ray diffraction pattern. Sometimes one or more peaks of intensity lower than 5% may be considered prominent and are used in addition with one or more peaks that are more prominent (i.e. at least about 10% or at least about 15% or at least about 20% relative intensity) in order to describe an XRPD pattern for a crystalline form of 3α-tetrol.

For identifying a crystalline form of 3α-tetrol in a solid formulation or invention composition, such as a tablet or in capsule granules, pairwise distribution function plots of principal components as described in US Pat. Pub. No. 2007/0110214 (which is incorporated by reference herein) are linearly combined and compared with the pairwise distribution plot of the solid formulation or invention composition. If the crystalline tetrol compound has been ground or micronized to such an extent that excessive line broadening prevents acquisition of meaningful XRPD data, precession electron diffraction using transmission electron microscopy as described in US Pat. Appl. No. 2007/0023659 (which is incorporated by reference herein) may be used as an alternative diffraction technique for identification of the crystalline form in such solid mixtures.

Vibrational Spectroscopy—

Diagnostic techniques that one can optionally use to characterize crystalline forms of 3α-tetrol include vibrational spectroscopy techniques such as IR and Raman, which measure the effect of incident energy on a solid state sample due to the presence of particular chemical bonds within molecules of the sample that vibrate in response to the incident energy. Since the molecules in different polymorphs experience different intermolecular forces due to variations in conformational or environmental factors, perturbations of those vibrations occur that leads to differences in spectra due to differences in frequency and intensity of some modes of vibration. Because polymorphs may possess different IR and Raman characteristics, IR and Raman spectrum provide complementary information and either may provide a fingerprint for identification of a particular polymorph. [see, Anderton, C. European Pharmaceutical Review, 9:68-74 (2004)].

In contrast to IR spectroscopy, Raman spectroscopy relies upon measuring light scattered from incident radiation of a particular wavelength directed to the sample, which can range from the UV to the near-IR. The light scattered contains not only photons with the same frequency as that of the incident radiation (called Rayleigh scattered light, which is filtered out), but also photons with a shifted frequency due to inelastic collisions with molecules within the solid state sample and it is these shifted frequencies that are determined by Raman spectroscopy. Thus, Raman scattered light is frequency-shifted (Raman-shift) with respect to the excitation frequency, but the magnitude of the shift is independent of the excitation frequency. Because Raman scattered light changes in frequency, the rule of conservation of energy dictates that some energy is deposited in the sample. Thus, a Raman shift will correspond to the excitation energy of a particular free vibration of a molecule in the solid state sample and is therefore an intrinsic property of the sample. While some molecular vibrations may be observed in both the IR and Raman spectra, sometimes vibrations observed using one technique will be weak or completely absent in the other due to the different fundamental physics underlying the two techniques.

Sample preparation in Raman spectroscopy is minimal, and If sample is limited it may be dispersed in oil or mixed with KBr to give enough material for introduction into the Raman spectrophotometer. Raman is also capable of determining polymorph identity or quantification in a complex matrix, distinguishing between non-crystalline and crystalline forms and is capable of differentiating between multiple polymorphic and pseudo polymorphic forms [for example, see Pratiwia, D., et al. “Quantitative analysis of polymorphic mixtures of ranitidine hydrochloride by Raman spectroscopy and principal components analysis” Eur. J. Pharm. Biopharm. 54(3), 337-341(2002)]. These capabilities are possible because features in the Raman are sharp and well resolved, which limits interference from excipients, and are very sensitive to the polymorphic form of a compound, with substantial spectral differences often evident between polymorphs, which also facilitates identification of more subtle features that may indicate the presence of a low level of one polymorph within another. For identifying a polymorphic form in a solid formulation such as a tablet, powder samples of 3α-tetrol in pure polymorphic form and excipients are gently compacted and scanned with Raman microscopy to build up a library of formulation component spectra. A partial least squares (PLS) model and multivariate classification are then used to analyze Raman mapping data obtained from sectioned tablets having low API content (about 0.5% w/w). Multivariate classification allows polymorph assignments to be made on individual microscopic pixels of 3α-tetrol identified in the data. By testing data from separate sets of tablets containing each polymorph, specific form recognition may be demonstrated at about 0.5% w/w. For tablets containing a mixture of forms, recognition of about 10% polymorphic impurity in a 3α-tetrol form (representing an absolute detection limit of about 0.05% w/w), is possible.

Overlap of IR bands from different solid state forms using the various methods described above sometimes occurs so that quantification requires deconvolution methods to extract information for each individual component in a mixture of crystalline forms. Such methods include partial least squares regression, principle component analysis or other methodologies [for examples, see Reich, G. “Near-infrared spectroscopy and imaging: Basic principles and pharmaceutical applications” Adv. Drug Deliv. Rev. 57: 1109-43 (2005)].

In one embodiment an amorphous form of 3α-tetrol is characterized by its XRPD and a spectrum obtained from a vibrational spectroscopy method, with Raman spectroscopy preferred.

Thermo Analysis Procedures—

Diagnostic techniques that one can optionally use to characterize a crystalline form of 3α-tetrol include differential thermal analysis (DTA), differential scanning calorimetry (DSC), thermo-gravimetric analysis (TGA) and melting point measurements.

DTA and DSC measures thermal transition temperatures at which a crystalline sample absorbs or releases heat when its crystal structure changes or it melts. TGA is used to measure thermal stability and the fraction of volatile components of a sample by monitoring the weight change as the sample is heated. These techniques are thus useful for characterizing crystalline forms existing as solvates and/or hydrates (i.e., pseudo-polymorphs).

DTA involves heating a test sample and an inert reference under identical conditions while recording any temperature difference between the sample and reference. This differential temperature is plotted against temperature, and changes in the test sample that leads to absorption or liberation of heat can thus be determined relative to the inert sample.

DSC measures the energy needed to establish a nearly zero temperature difference between a sample and an inert reference as they are subjected to identical heating regimes. In power compensated DSC, the temperatures of the sample and reference are controlled independently. The temperature of the sample and reference are made identical by varying the power input of the two heaters in which the sample and reference reside. The energy required to do this is a measure of the enthalpy or heat capacity changes in the sample relative to the reference.

Transition temperatures in DSC or DTA for sharply-defined endotherms or exotherms typically occur within about 4° C. on successive analyses of crystalline 3α-tetrol samples using a temperature scan rate of 10° C./min. Thus, when a crystalline form of 3α-tetrol is reported to have a thermal transition at a given value, it means that the DTA or DSC transition is within ±2° C. of the reported value. Different crystalline forms may be identified, at least in part, based on their different transition temperature profiles in their DTA or DSC thermographs and optionally based upon their weight loss in TGA within a defined temperature range.

### NUMBERED EMBODIMENTS

The following embodiments exemplify one or more aspects of the invention are not meant to be limiting in any way.

### Numbered Embodiments

The following embodiments exemplify or describe one or more aspects of the invention are not meant to be limiting in any way.

1. Crystalline androst-5-ene-3α,7β,16α,17β-tetrol.

2. The crystalline androst-5-ene-3α,7β,16α,17β-tetrol of embodiment 1 wherein crystalline androst-5-ene-3α,7β,16α,17β-tetrol is a crystalline anhydrate.

3. The crystalline androst-5-ene-3α,7β,16α,17β-tetrol of embodiment 1 wherein crystalline androst-5-ene-3α,7β,16α, 17β-tetrol is Form Iα or Form IIα 3α-tetrol.

4. The crystalline androst-5-ene-3α,7β,16α,17β-tetrol of embodiment 3 wherein crystalline androst-5-ene-3α,7β,16α,17β-tetrol is Form Iα 3α-tetrol characterized by (1) an XRPD pattern having three or more peaks selected from the group consisting of about 7.6, 16.1, 17.8, 19.8 and 22.2 degree 2-theta and optionally one or more peaks selected from the group consisting of about 13.7, 15.3, 16.5, 17.0 and 20.9 degree 2-theta or (2) DTA thermogram, obtained with a temperature ramp of 10° C./min, having a prominent endotherm centered at about 224° C., optionally having an onset temperature of about 216° C., and an exotherm centered at about 154° C. or (3): (1) and (2).

Form Iα 3α-tetrol can be characterized by (a) an XRPD pattern with three peaks at about 7.6, 16.1 and 17.8, about 7.6, 16.1 and 19.8, about 7.6, 16.1 and 22.2, about 16.1, 17.8 and 19.8, about 16.1, 17.8 and 22.2, or about 17.8, 19.8 and 22.2 degree 2-theta, (b) one XRPD peak at about 13.7, 15.3, 16.5, 17.0 or 20.9 degree 2-theta and (c) optionally a DTA thermogram, obtained with a temperature ramp of 10° C./min, having a prominent endotherm centered at about 224° C., optionally having an onset temperature of about 216° C., and an exotherm centered at about 154° C.

Form Iα 3α-tetrol can also be characterized by (a) an XRPD pattern with peaks at about 7.6, 16.1, 17.8 and 19.8, about 7.6, 16.1, 17.8 and 22.2 or about 7.6, 16.1, 17.8, 19.8 and 22.2 degree 2-theta, (b) one XRPD peak at about 13.7, 15.3, 16.5, 17.0 or 20.9 degree 2-theta and (c) optionally a DTA thermogram, obtained with a temperature ramp of 10° C./min, having a prominent endotherm centered at about 224° C., optionally having an onset temperature of about 216° C., and an exotherm centered at about 154° C. Form Iα 3α-tetrol can also be characterized by (a) an XRPD pattern with three peaks at about 7.6, 16.1 and 17.8, about 7.6, 16.1 and 19.8, about 7.6, 16.1 and 22.2, about 16.1, 17.8 and 19.8, about 16.1, 17.8 and 22.2, or about 17.8, 19.8 and 22.2 degree 2-theta, (b) two XRPD peaks at about 13.7 and 15.3, about 13.7 and 16.5, about 13.7 and 17.0, about 13.7 and 20.9, about 15.3 and 16.5, about 15.3 and 17.0 or about 15.3 and 20.9 degree 2-theta and (c) optionally a DTA thermogram, obtained with a temperature ramp of 10° C./min, having a prominent endotherm centered at about 224° C., optionally having an onset temperature of about 216° C., and an exotherm centered at about 154° C.

Form Iα 3α-tetrol can also be characterized by (a) an XRPD pattern with three peaks at about 7.6, 16.1 and 17.8, about 7.6, 16.1 and 19.8, about 7.6, 16.1 and 22.2, about 16.1, 17.8 and 19.8, about 16.1, 17.8 and 22.2, or about 17.8, 19.8 and 22.2 degree 2-theta, (b) two XRPD peaks at about 16.5 and 17.0 about 16.5 and 20.9 or about 17.0 and 20.9 degree 2-theta and (c) optionally a DTA thermogram, obtained with a temperature ramp of 10° C./min, having a prominent endotherm centered at about 224° C., optionally having an onset temperature of about 216° C., and an exotherm centered at about 154° C. Form Iα 3α-tetrol can also be characterized by (a) an XRPD pattern with peaks at about 7.6, 16.1, 17.8 and 19.8, about 7.6, 16.1, 17.8 and 22.2 or about 7.6, 16.1, 17.8, 19.8 and 22.2 degree 2-theta, (b) two XRPD peaks at about 13.7 and 15.3, about 13.7 and 16.5, about 13.7 and 17.0, about 13.7 and 20.9, about 15.3 and 16.5, about 15.3 and 17.0 or about 15.3 and 20.9 degree 2-theta and (c) optionally a DTA thermogram, obtained with a temperature ramp of 10° C./min, having a prominent endotherm centered at about 224° C., optionally having an onset temperature of about 216° C., and an exotherm centered at about 154° C.

Form Iα 3α-tetrol can also be characterized by (a) an XRPD pattern with peaks at about 7.6, 16.1, 17.8 and 19.8, about 7.6, 16.1, 17.8 and 22.2 or about 7.6, 16.1, 17.8, 19.8 and 22.2 degree 2-theta, (b) two XRPD peaks at about 16.5 and 17.0 about 16.5 and 20.9 or about 17.0 and 20.9 degree 2-theta and (c) optionally a DTA thermogram, obtained with a temperature ramp of 10° C./min, having a prominent endotherm centered at about 224° C., optionally having an onset temperature of about 216° C., and an exotherm centered at about 154° C.

5. The crystalline androst-5-ene-3α,7β,16α,17β-tetrol of embodiment 3 wherein crystalline androst-5-ene-3α,7β,16α,17β-tetrol is further characterized by TGA thermogram, obtained with a temperature ramp of 10° C./min, having negligible % weight loss or 2% wt loss from about 60° C. to 140° C. or from about 60° C. to onset of the prominent endotherm.

6. The crystalline androst-5-ene-3α,7β,16α,17β-tetrol of embodiment 1 wherein crystalline androst-5-ene-3α,7β,16α,17β-tetrol is Form IIα 3α-tetrol characterized by DTA thermogram, obtained with a temperature ramp of 10° C./min, having a prominent endotherm at centered about 243° C., optionally having an onset temperature of about 229° C. or a shoulder between about 230 to 240° C.

7. Amorphous androst-5-ene-3α,7β,16α,17β-tetrol.

8. The amorphous androst-5-ene-3α,7β,16α,17β-tetrol of embodiment 7 characterized by (1) XRPD pattern having a broad band from about 11 degree 2-theta to about 20 degree 2-theta or centered between about 16 to 17 degree 2-theta or (2) a DTA thermogram, obtained with a temperature ramp of 10° C./min, having a prominent exotherm centered at about 166° C. and a endotherm centered at about 225° C., optionally having a shoulder at about 220° C. or (3): (1) and (2).

9. The amorphous androst-5-ene-3α,7β,16α,17β-tetrol of embodiment 8 further characterized by TGA thermogram with variable weight loss from about 140° C. to about 240° C.

8. A composition comprising, consisting essentially of or consisting of one or more excipients and a solid state form of androst-5-ene-3α,7β,16α,17β-tetrol.

9. The composition of embodiment 8 wherein the solid state form is a crystalline anhydrate.

10. The composition of embodiment 9 wherein the crystalline anhydrate is Form Iα 3α-tetrol.

11. The composition of embodiment 9 wherein the crystalline anhydrate is Form IIα 3α-tetrol.

12. A method of preparing a liquid formulation comprising, consisting essentially of or consisting of admixing a solid state form of androst-5-ene-3α,7β,16α,17β-tetrol with a liquid excipient.

13. The method of embodiment 12 wherein the solid state form is a crystalline anhydrate.

14. The method of embodiment 13 wherein the crystalline anhydrate is Form Iα or Form IIα 3α-tetrol.

15. The method of embodiment 12 wherein the solid state form is amorphous 3α-tetrol.

16. A method of treating unwanted inflammation, comprising administering an effective amount of a solid formulation to a subject in need thereof wherein the solid formulation comprising, consisting essentially of or consisting of a solid state form of androst-5-ene-3α,7β,16α,17β-tetrol and one or more excipients.

17. The method of embodiment 16 wherein the solid state form is a crystalline anhydrate.

18. The method of embodiment 17 wherein the crystalline anhydrate is Form Iα or Form IIα 3α-tetrol.

19. The method of embodiment 16 wherein the solid state form is amorphous 3α-tetrol.

20. The method of embodiment 16 wherein the unwanted inflammation is a condition or disease associated with chronic, non-production inflammation.

21. The method of embodiment 16 wherein the condition or disease is an autoimmune condition or disease.

22. The method of embodiment 16 wherein the condition or disease is a metabolic condition or disease.

23. The method of embodiment 21 wherein the autoimmune disease is Type 1 diabetes.

24. The method of embodiment 21 wherein the autoimmune disease is a lupus condition such as systemic lupus erythematosus or discoid lupus or an arthritis condition such as rheumatoid arthritis.

25. The method of embodiment 16 wherein the condition or disease is an inflammatory bowel disease such as ulcerative colitis or Crohn's disease (regional enteritis).

26. The method of embodiment 16 wherein the condition or disease is a lung inflammation condition such as cystic fibrosis, chronic obstructive pulmonary disease (COPD), acute asthma, chronic asthma, emphysema, acute bronchitis, allergic bronchitis, chronic bronchitis and fibrosing alveolitis (lung fibrosis) conditions, e.g., subepithelial fibrosis in patients having chronic bronchitis, asthma and/or COPD.

27. The method of embodiment 16 wherein the condition or disease is a neurodegenerative condition such as Parkinson's disease or Alzheimer's disease.

28. The method of embodiment 16 wherein the condition or disease is a hyperproliferation condition.

29. The method of embodiment 16 wherein the condition or disease is a liver cirrhosis condition, nonalcoholic steatohepatitis (NASH) or fatty liver conditions.

30. The method of embodiment 22 wherein the metabolic condition or disease is type 2 diabetes, obesity, insulin resistance, hyperglycemia, impaired glucose utilization or tolerance, impaired or reduced insulin synthesis.

1A. A product wherein the product is a solid state form of androst-5-ene-3α,7β,16α,17β-tetrol obtained by the process comprising, consisting essentially of or consisting of (1) admixing 3α-tetrol with a volume of acetone at a temperature between room temperature and the boiling point of acetone at ambient pressure to provide a suspension; and (2) agitating the suspension of 3α-tetrol with heating wherein a majority of the initial mass of 3α-tetrol of said admixing remains in suspension.

2A. The product of embodiment 1A wherein the acetone suspension of said agitating is heated to a temperature between about 40° C. to about the boiling point of the acetone suspension at ambient pressure.

3A. The product of embodiment 1A wherein the acetone suspension of said agitating is heated to the boiling point at ambient pressure wherein acetone in said admixing is used in sufficient volume to dissolve between about 5% to about 25% of the initial mass of the 3α-tetrol.

4A. The product of embodiment 4A wherein 3α-tetrol of said suspension is amorphous 3α-tetrol.

5A. The product of embodiment 4A wherein the amorphous 3α-tetrol is prepared according to Example 5.

6A. A product wherein the product is a solid state form of androst-5-ene-3α,7β,16α,17β-tetrol obtained by the process comprising, consisting essentially of or consisting of (1) mixing 3α-tetrol with a volume of acetone at a temperature sufficient to provide a homogeneous solution; and (2) reducing the temperature of the solution to a temperature wherein a majority of the initial mass of 3α-tetrol of said mixing precipitates on standing at the reduced temperature.

7A. The product of embodiment 6A wherein the temperature of the homogeneous solution from said mixing is at or near the boiling point of the solution at ambient pressure.

8A. The product of embodiment 6A wherein the temperature of the homogeneous solution is reduced to room temperature.

9A. The product of embodiment 5A wherein the acetone solution of said mixing is of a acetone volume that results in precipitation of the majority of the initial mass of 3α-tetrol with said temperature reducing on standing at the reduced temperature.

10A. The product of embodiment 5A wherein the acetone solution of said mixing is the minimum acetone volume to provide a homogeneous solution upon heating at or near the boiling point of acetone.

11A. The product of embodiment 9A or 10A wherein 3α-tetrol of said mixing is prepared according to Example 1.

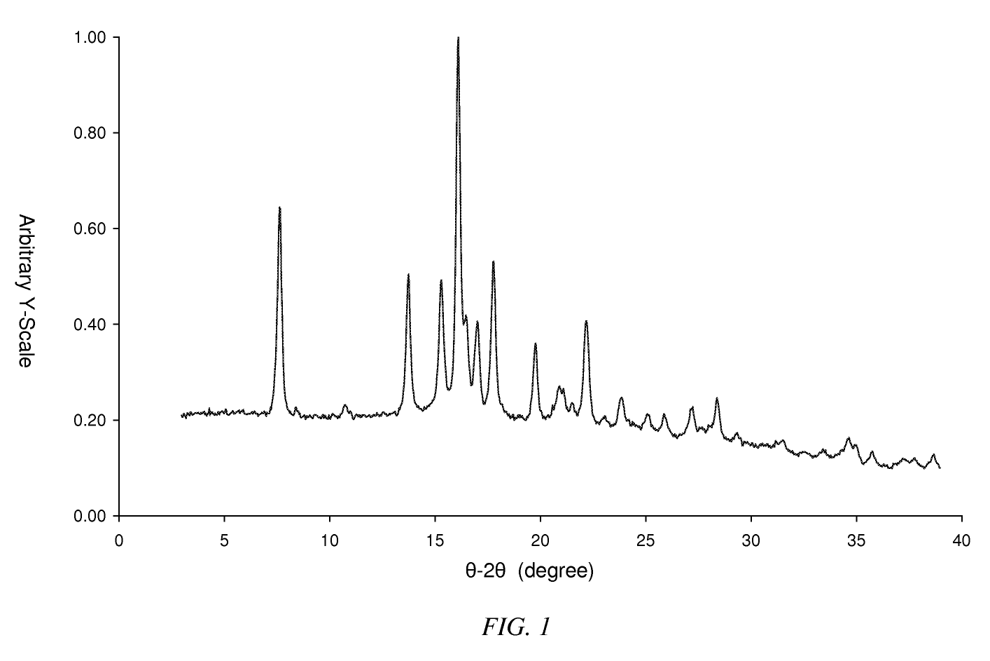

12A. The product of embodiment 1A or 6A characterized by an X-ray powder pattern substantially identical to FIG. 1.

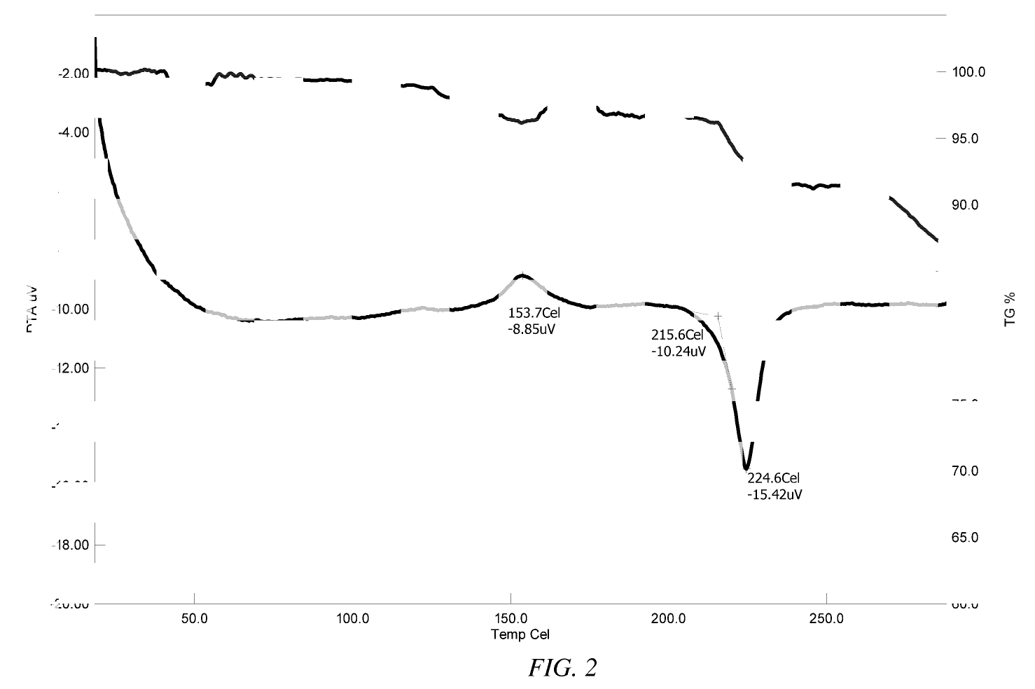

13A. The product of embodiment 1A or 6A wherein the solid state form is characterized by DTA-TG thermogram traces substantially identical to FIG. 2 and optionally with one, two three or more prominent XRPD peaks of Table 1.

14A. A composition comprising one or more excipients and a solid state form of androst-5-ene-3α,7β,16α,17β-tetrol obtained by the process of embodiment 1A or 6A.

15A. A method of preparing a liquid formulation comprising, consisting essentially of or consisting of admixing a solid state form of androst-5-ene-3β7β,16α,17β-tetrol, obtained by the process of embodiment 1A or 6A.

16A. A method of treating an inflammation condition or disease or another condition or disease described herein, comprising administering an effective amount of a solid formulation to a subject in need thereof wherein the formulation comprises, consisting essentially of or consists of a solid state form of androst-5-ene-3β,7β,16α,17β-tetrol, obtained by the process of embodiment 1A or 6A, and one or more excipients.

17A. The method of embodiment 16A wherein the inflammation condition or disease is associated with chronic, non-production inflammation.

18A. The method of embodiment 16A wherein the condition or disease is an autoimmune condition or disease.

19A. The method of embodiment 16A wherein the condition or disease is a metabolic condition or disease.

20A. The method of embodiment 18A wherein the autoimmune disease is Type 1 diabetes.

21A. The method of embodiment 18A wherein the autoimmune disease is a lupus condition such as systemic lupus erythematosus or discoid lupus or an arthritis condition such as rheumatoid arthritis.

22A. The method of embodiment 16A wherein the condition or disease is an inflammatory bowel disease such as ulcerative colitis or Crohn's disease (regional enteritis).

23A. The method of embodiment 16A wherein the condition or disease is a lung inflammation condition such as cystic fibrosis, chronic obstructive pulmonary disease (COPD), acute asthma, chronic asthma, emphysema, acute bronchitis, allergic bronchitis, chronic bronchitis and fibrosing alveolitis (lung fibrosis) conditions, e.g., subepithelial fibrosis in patients having chronic bronchitis, asthma and/or COPD.

24A. The method of embodiment 16A wherein the condition or disease is a neurodegenerative condition such as Parkinson's disease or Alzheimer's disease.

25A. The method of embodiment 16A wherein the condition or disease is a hyperproliferation condition.

26A. The method of embodiment 16A wherein the condition or disease is a liver cirrhosis condition, nonalcoholic steatohepatitis (NASH) or fatty liver conditions.

27A. The method of embodiment 19A wherein the metabolic condition or disease is type 2 diabetes, obesity, insulin resistance, hyperglycemia, impaired glucose utilization or tolerance, impaired or reduced insulin synthesis.

28A. A product wherein the product is a solid state form of androst-5-ene-3β,7β,16α,17β-tetrol obtained by the process comprising, consisting essentially of or consisting of (1) mixing 3α-tetrol in a volume of ethanol to provide an ethanolic solution at ambient temperature (2) mixing the ethanolic solution with a volume of diethyl ether at ambient temperature; and (3) reducing the temperature of the ethanol-diethyl ether admixture to a temperature wherein at least 5% of the initial mass of 3α-tetrol of said ethanol mixing precipitates on standing at the reduced temperature.

29A. The product of embodiment 28A wherein the ethanol is denatured ethanol having a water content of about 5% by volume or less.

31A. The product of embodiment 28A wherein the concentration of androst-5-ene-3β,7β,16α,17β-tetrol in the ethanolic solution is between about 200 mg/mL to about 300 mg/mL.

32A. The product of embodiment 31A wherein ethanol and ethyl ether of said mixings are of about equal volume.

33A. The product of embodiment 38A wherein the ethanol-diethyl ether admixture temperature reduction is to between about −10° C. to about −20° C. and between about 10% to about 20% of the initial mass of 3α-tetrol of said ethanol admixing precipitates on standing at the reduced temperature after between about 1 day to about 10 days.

34A. The product of embodiment 33A wherein the 3α-tetrol of said ethanol admixing is prepared according to Example 4.

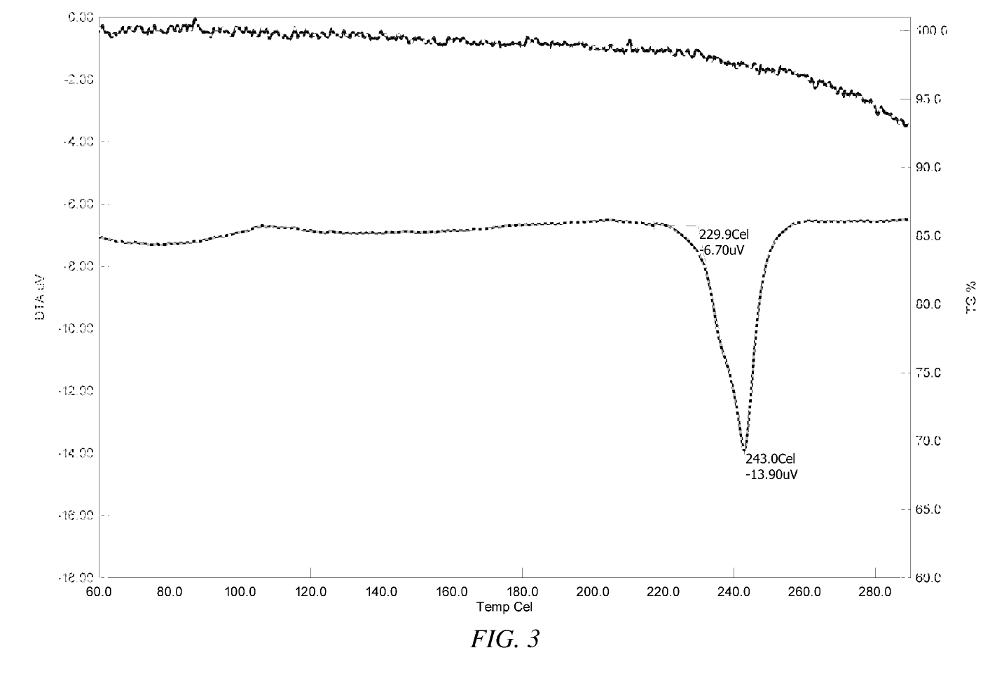

35A. The product of embodiment 28A wherein the solid state form is characterized by DTA-TG thermogram traces substantially identical to FIG. 3.

36A. A composition comprising one or more excipients and a solid state form of androst-5-ene-3β,7β,16β,17β-tetrol obtained by the process of embodiment 28A.

37A. A method of preparing a liquid formulation comprising admixing a solid state form of androst-5-ene-3β,7β,16α,17β-tetrol, obtained by the process of embodiment 28A.

38A. A method of treating an inflammation condition or disease or another condition or disease described herein, comprising administering an effective amount of a solid formulation to a subject in need thereof wherein the formulation comprises, consisting essentially of or consists of a solid state form of androst-5-ene-3β,7β,16α,17β-tetrol, obtained by the process of embodiment 28A, and one or more excipients.

39A. The method of embodiment 38A wherein the inflammation condition or disease is associated with chronic, non-production inflammation.

40A. The method of embodiment 38A wherein the condition or disease is an autoimmune condition or disease.

41A. The method of embodiment 38A wherein the condition or disease is a metabolic condition or disease.

42A. The method of embodiment 40A wherein the autoimmune disease is Type 1 diabetes.

43A. The method of embodiment 40A wherein the autoimmune disease is a lupus condition such as systemic lupus erythematosus or discoid lupus or an arthritis condition such as rheumatoid arthritis.

44A. The method of embodiment 38A wherein the condition or disease is an inflammatory bowel disease such as ulcerative colitis or Crohn's disease (regional enteritis).

45A. The method of embodiment 38A wherein the condition or disease is a lung inflammation condition such as cystic fibrosis, chronic obstructive pulmonary disease (COPD), acute asthma, chronic asthma, emphysema, acute bronchitis, allergic bronchitis, chronic bronchitis and fibrosing alveolitis (lung fibrosis) conditions, e.g., subepithelial fibrosis in patients having chronic bronchitis, asthma and/or COPD.

46A. The method of embodiment 38A wherein the condition or disease is a neurodegenerative condition such as Parkinson's disease or Alzheimer's disease.

47A. The method of embodiment 38A wherein the condition or disease is a hyperproliferation condition.

48A. The method of embodiment 38A wherein the condition or disease is a liver cirrhosis condition, nonalcoholic steatohepatitis (NASH) or fatty liver conditions.

49A. The method of embodiment 41A wherein the metabolic condition or disease is type 2 diabetes, obesity, insulin resistance, hyperglycemia, impaired glucose utilization or tolerance, impaired or reduced insulin synthesis.

1B. A product wherein the product is a solid state form of androst-5-ene-3β,7β,16α,17β-tetrol obtained by the process comprising, consisting essentially of or consisting of (1) lyophilizing a mixture 3α-tetrol in an methanol-water solvent mixture to provide a first solid 3α-tetrol material, (2) filtering a THF solution of the first solid 3α-tetrol material from said lyophilizing to remove un-dissolved components, (3) recovering a second solid 3α-tetrol material from filtrate of said filtering; and (4) admixing a methanolic solution of the second solid 3α-tetrol material from said recovering with diethyl ether.

2B. The product of embodiment 1B wherein 3α-tetrol of said lyophilization is prepared according to example 1.

3B. The product of embodiment 1B wherein the methanol to water ratio of the methanol-water solvent mixture is 10:1 v/v.

3B The product of embodiment 1B wherein the ratio of 3α-tetrol to THF in filtrate from said filtering is between about 25 mg/mL to about 50 mg/mL.

4B. The product of embodiment 3B wherein said recovering is precipitating the second solid 3α-tetrol material by admixing the THF filtrate with methanol.

5B. The product of embodiment 3B wherein said admixing of the methanolic solution of the second solid 3α-tetrol material is by slow addition of ethyl ether to the methanolic solution.

6B. The product of embodiment 1B wherein said steps are conducted according to Example 5.

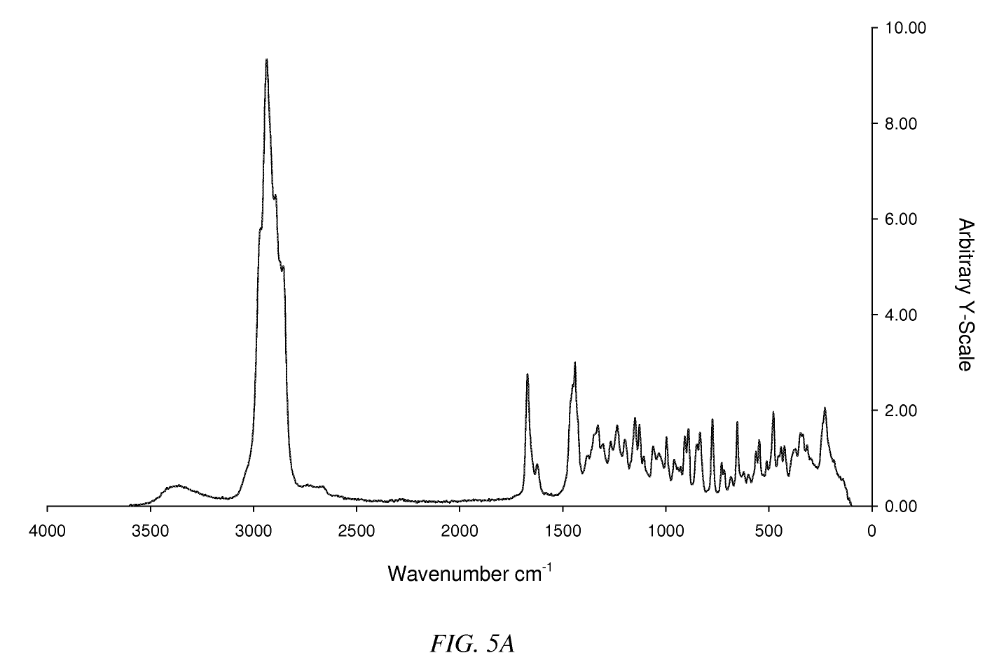

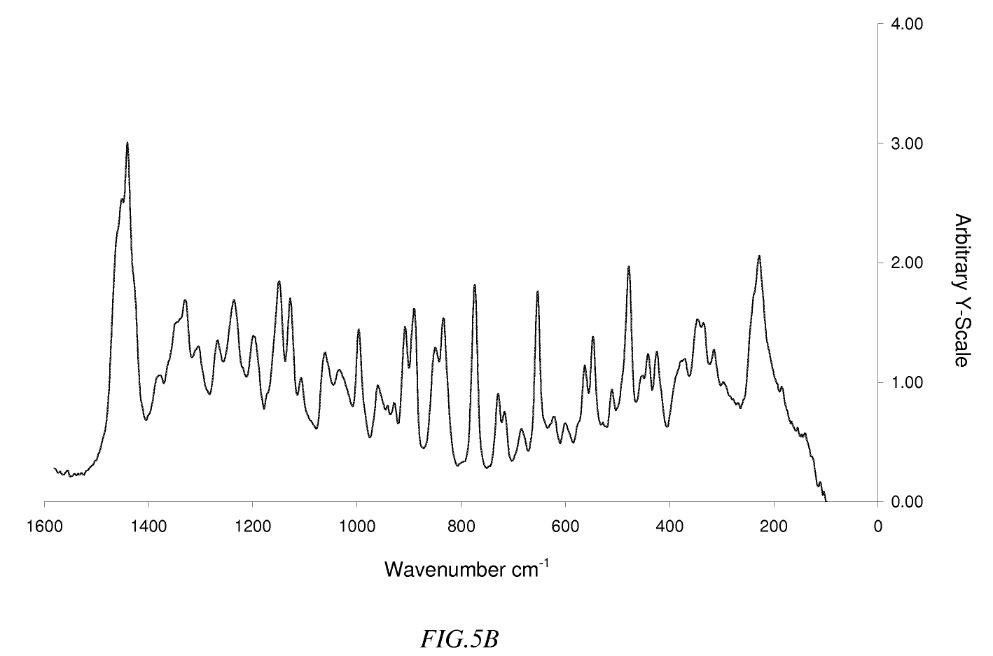

7B. The product of embodiment 1B characterized by a solid infrared Raman spectrum substantially identical to FIG. 5A or 5B.

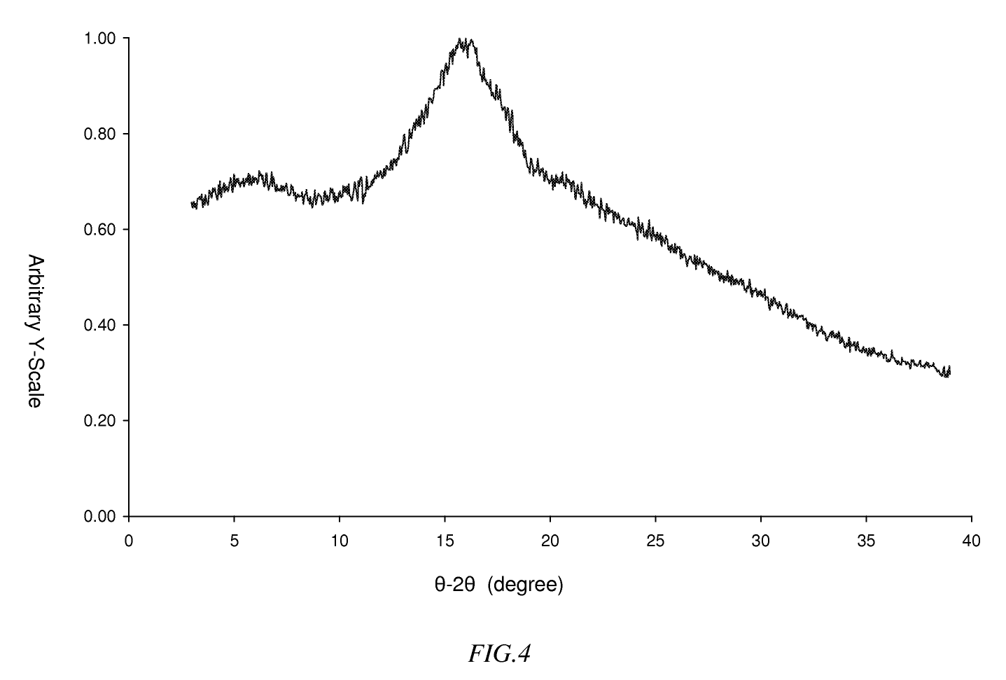

8B. The product of embodiment 1B characterized by an X-ray powder pattern substantially identical to FIG. 4 and a solid infrared Raman spectrum substantially identical to FIG. 5B.

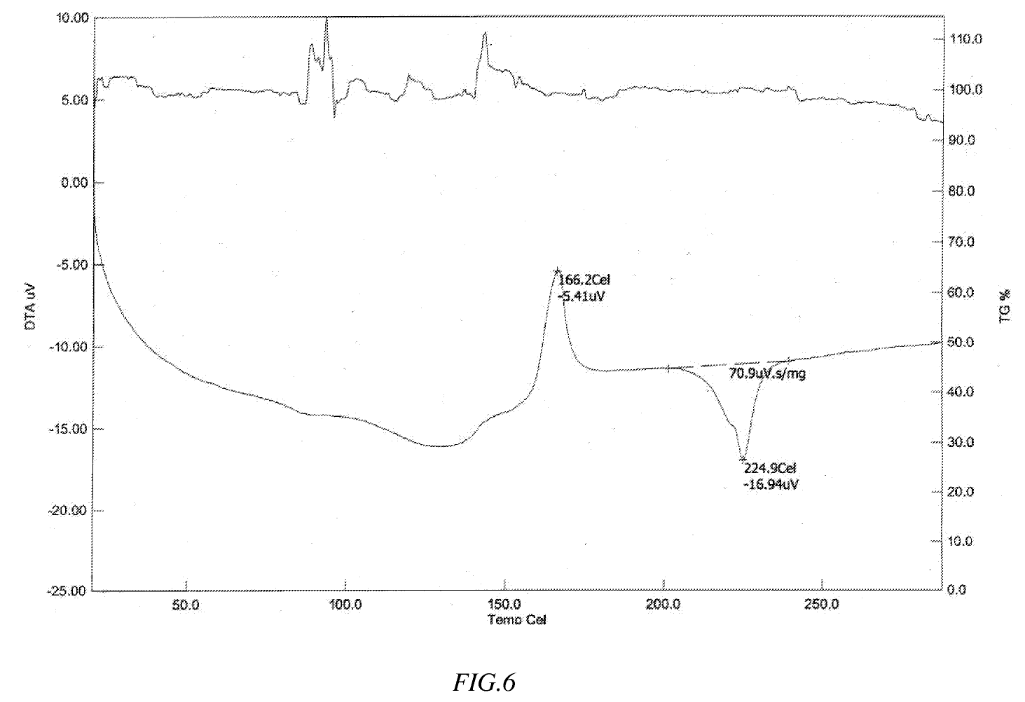

9B. The product of embodiment 1B wherein the solid state form is characterized by DTA-TG thermogram traces substantially identical to FIG. 6 and optionally a solid infrared Raman spectrum substantially identical to FIG. 5B.

10B. A composition comprising, consisting essentially of or consisting of one or more excipients and a solid state form of androst-5-ene-3α,7β,16α,17β-tetrol obtained by the process of embodiment 1B.

11B. A method of preparing a liquid formulation comprising, consisting essentially of or consisting of admixing a solid state form of androst-5-ene-3α,7β,16α,17β-tetrol, obtained by the process of embodiment 1B.

12B. A method of treating an inflammation condition or disease or another condition or disease described herein, comprising administering an effective amount of a solid formulation to a subject in need thereof wherein the formulation comprises, consists essentially of or consists of a solid state form of androst-5-ene-3β,7β,16α,17β-tetrol, obtained by the process of embodiment 1B, and one or more excipients.

13B. The method of embodiment 12B wherein the inflammation condition or disease is associated with chronic, non-production inflammation.

14B. The method of embodiment 12B wherein the condition or disease is an autoimmune condition or disease.

15B. The method of embodiment 12B wherein the condition or disease is a metabolic condition or disease.

16B. The method of embodiment 14B wherein the autoimmune disease is Type 1 diabetes.

17B. The method of embodiment 14B wherein the autoimmune disease is a lupus condition such as systemic lupus erythematosus or discoid lupus or an arthritis condition such as rheumatoid arthritis.

18B. The method of embodiment 12B wherein the condition or disease is an inflammatory bowel disease such as ulcerative colitis or Crohn's disease (regional enteritis).

19B. The method of embodiment 12B wherein the condition or disease is a lung inflammation condition such as cystic fibrosis, chronic obstructive pulmonary disease (COPD), acute asthma, chronic asthma, emphysema, acute bronchitis, allergic bronchitis, chronic bronchitis and fibrosing alveolitis (lung fibrosis) conditions, e.g., subepithelial fibrosis in patients having chronic bronchitis, asthma and/or COPD.

20B. The method of embodiment 19B wherein the condition or disease is a neurodegenerative condition. The method of embodiment 19B wherein the neurodegenerative condition is Parkinson's disease. The method of embodiment 19B wherein the neurodegenerative condition is Alzheimer's disease.

21B. The method of embodiment 12B wherein the condition or disease is a hyperproliferation or cancer condition. In some of these embodiments the hyperproliferation or cancer condition is prostate cancer. In some of these embodiments the hyperproliferation or cancer condition is breast cancer. In some of these embodiments the hyperproliferation or cancer condition is endometriosis.

22B. The method of embodiment 12B wherein the condition or disease is a liver cirrhosis condition, NASH, NAFLD or other fatty liver conditions.

23B. The method of embodiment 15B wherein the metabolic condition or disease is type 2 diabetes, obesity, insulin resistance, hyperglycemia, impaired glucose utilization or tolerance, impaired or reduced insulin synthesis.

10. A solid state form of 3α-tetrol wherein 3α-tetrol is (a) a powder or granule that is at least 80% pure, at least 95% pure or at least 98% pure or (b) a solution or suspension that is at least 80% pure, at least 95% pure or at least 98% pure.

2C. The solid state form of embodiment 21C wherein 3α-tetrol is about 80%, about 85%, about 90%, about 95%, about 97% or about 98% to about 99.5% or about 99.9% pure, optionally wherein 3α-tetrol is in the form of a powder or granules, optionally wherein the powder has an average particle size of about 50 nm or about 100 nm to about 5 μm, about 10 μm or about 25 μm as measured in a suitable assay such as light scattering.

3C. A method of treatment or prophylaxis of an autoimmune disease or unwanted inflammation condition, which optionally is an arthritis condition such as an osteoarthritis (primary or secondary osteoarthritis), rheumatoid arthritis, an arthritis associated with spondylitis such as ankylosing spondylitis, multiple sclerosis, Alzheimer's disease, a lupus condition such as systemic lupus erythematosis or discoid lupus erythematosis, tendinitis, bursitis, a lung inflammation condition such as asthma, emphysema, chronic obstructive pulmonary disease, lung fibrosis, cystic fibrosis, acute or adult respiratory distress syndrome, chronic bronchitis, acute bronchitis, bronchiolitis, bronchiolitis fibrosa obliterans, bronchiolitis obliterans with organizing pneumonia, using 3α-tetrol as Form Iα, Form IIα or amorphous 3α-tetrol or a mixture thereof.

4C. The method of embodiment 3C comprising administering to the human or the rodent a treatment effective amount of 3α-tetrol. Such treatments include treatment with about 0.1 mg/day, about 1 mg/day or about 5 mg/day to about 40 mg/day or about 80 mg/day of 3α-tetrol.

5C. The method of embodiment 3C wherein the autoimmune or related disorder is ulcerative colitis, inflammatory bowel disease, Crohn's disease, psoriasis, actinic keratosis, arthritis, multiple sclerosis, optic neuritis or a dermatitis condition, optionally contact dermatitis, atopic dermatitis or exfoliative dermatitis.

1D. A crystalline form or a composition or formulation comprised of a crystalline form of 3α-tetrol wherein the crystalline form is characterized by (1) an XRPD pattern having four, five or more peaks selected from the group consisting of 7.6±0.1, 13.7±0.1, 15.3±0.1, 16.1±0.1, 16.5±0.1, 17.0±0.1, 17.8±0.1, 19.8±0.1, 20.9±0.1, 21.1±0.1, 22.2±0.1, 27.2±0.1 and 28.4±0.1 degree 2-theta or (2) DTA thermogram, obtained with a temperature ramp of about 10° C./min, having a prominent endotherm centered at about 225° C. and an exotherm centered at about 154° C. or (3): (1) and (2).

2D. A crystalline form or a composition or formulation comprised of a crystalline form wherein the crystalline form is characterized by (1) XRPD pattern having four or more prominent peaks of Table 1, wherein the prominent peaks have at least 10% relative intensity and (2) DTA thermogram, obtained with a temperature ramp of about 10° C./min, having a prominent endotherm centered at about 225° C. and an exotherm centered at about 154° C.

3D. The crystalline form of embodiment 1D or 2D wherein the crystalline form is further characterized by TGA thermogram obtained with a temperature ramp of about 10° C./min, having negligible % weight loss from about 80° C. to about 120° C. or less than about 4% weight loss from about 80° C. to about 160° C.

4D. Amorphous androst-5-ene-3α,7β,16α,17β-tetrol, or a composition or formulation comprised of androst-5-ene-3α,7β,16α,17β-tetrol in amorphous form wherein the crystalline form is characterized by (1) a solid state Raman spectrum having two or more absorbances selected from the group consisting of 316, 337, 349, 372, 380, 426, 443 and 453 cm⁻¹ and two or more absorbances selected from the group consisting of 910, 1109, 1120, 1151, 1201, 1238, 1269, 1306, 1333, 1381 and 1442 cm⁻¹, optionally having an absorbance at 2969 cm⁻¹ or (2) DTA thermogram, obtained with a temperature ramp of about 10° C./min, having a prominent endotherm centered at about 225° C. and an exotherm centered at about 154° C. or (3): (1) and (2).

5D. The amorphous androst-5-ene-3α,7β,16α,17β-tetrol form of embodiment 3D or 4D wherein the crystalline form is further characterized by TGA thermogram obtained with a temperature ramp of about 10° C./min, having negligible % weight loss from about 80° C. to about 120° C. and/or less than about 4% weight loss from about 80° C. to about 160° C.

6D. A crystalline form or a composition or formulation comprised of a crystalline form of 3α-tetrol wherein the crystalline form is characterized by (1) an XRPD pattern having four, five or more peaks selected from the group consisting of 7.6±0.2, 13.7±0.2, 15.3±0.2, 16.1±0.2, 16.5±0.2, 17.0±0.2, 17.8±0.2, 19.8±0.2, 20.9±0.2, 21.1±0.2, 22.2±0.2, 27.2±0.2 and 28.4±0.2 degree 2-theta or (2) DTA thermogram, obtained with a temperature ramp of about 10° C./min, having a prominent endotherm centered at about 225° C. and an exotherm centered at about 154° C. or (3): (1) and (2).

1E. The crystalline androst-5-ene-3α,7β,16α,17β-tetrol of embodiment 1 wherein crystalline androst-5-ene-3α,7β,16α,17β-tetrol is Form Iα 3α-tetrol characterized by (1) an XRPD pattern having three or more peaks selected from the group consisting of 7.6, 16.1, 17.8, 19.8 and 22.2 degree 2-theta and one or more peaks selected from the group consisting of 13.7, 15.3, 16.5, 17.0 and 20.9 degree 2-theta or (2) DTA thermogram, obtained with a temperature ramp of 10° C./min, having a prominent endotherm centered at 224° C. or (3): (1) and (2).

2E. The crystalline androst-5-ene-3α,7β,16α,17β-tetrol of embodiment 1E or 2E, wherein the 224° C. DTA endotherm has an onset temperature of 216° C.

3E. The crystalline androst-5-ene-3α,7β,16α,17β-tetrol of embodiment 1E or 2E, wherein the DTA thermogram further has an exotherm centered at about 154° C.

4E. The crystalline androst-5-ene-3α,7β,16α,17β-tetrol of 1E, 2E or 3E wherein crystalline androst-5-ene-3α,7β,16α,17β-tetrol is further characterized by TGA thermogram, obtained with a temperature ramp of 10° C./min, having negligible % weight loss from about 60° C. to 140° C. and/or 2% wt loss from about 60° C. to the onset of the 224° C. DTA endotherm.

5E. The crystalline androst-5-ene-3α,7β,16α,17β-tetrol of embodiment 1 wherein crystalline androst-5-ene-3α,7β,16α,17β-tetrol is Form IIα 3α-tetrol characterized by DTA thermogram, obtained with a temperature ramp of 10° C./min, having a prominent endotherm centered at about 243° C.

6E. The crystalline androst-5-ene-3α,7β,16α,17β-tetrol of embodiment 5E wherein the 243° C. DTA endotherm has an onset temperature of about 229° C. and/or a shoulder between about 230 to 240° C.

7E. The amorphous androst-5-ene-3α,7β,16α,17β-tetrol of embodiment 7 wherein the amorphous androst-5-ene-3α,7β,16α,17β-tetrol is characterized by (1) XRPD pattern having a broad band from 11 degree 2-theta to 20 degree 2-theta or a broad band centered between 16 to 17 degree 2-theta or (2) DTA thermogram, obtained with a temperature ramp of 10° C./min, having an prominent exotherm centered at 166° C. or (3): (1) and (2).

8E. The amorphous androst-5-ene-3α,7β,16α,17β-tetrol of embodiment 7E wherein the DTA thermogram further has a prominent enotherm centered at about 225° C.

9E. The amorphous androst-5-ene-3α,7β,16α,17β-tetrol of embodiment 8E wherein the 225° C. DTA endothern has a shoulder at 220° C.

10E. The amorphous androst-5-ene-3α,7β,16α,17β-tetrol of embodiment 8 further characterized by TGA thermogram with negligible % weight loss from 60° C. to 140° C.

8E. A composition comprising, consisting essentially or consisting of one or more excipients and a solid state form of androst-5-ene-3α,7β,16α,17β-tetrol wherein the solid state form is crystalline androst-5-ene-3α,7β,16α,17β-tetrol.

9E. The composition of embodiment 8E wherein the crystalline androst-5-ene-3α,7β,16α,17β-tetrol form is a crystalline anhydrate.

10E. The composition of embodiment 9E wherein the crystalline anhydrate is Form Iα 3α-tetrol.

11E. The composition of embodiment 9E wherein the crystalline anhydrate is Form IIα 3α-tetrol.

For any one of embodiments 1E to 11E where XRDP and/or solid stae Raman and/or DTA thermal data is recited therein, such data typically have uncertanties of ±0.2 degress 2-theta, ±2 cm⁻¹ and ±2° C. for prominent exotherm and prominent endotherm inflection points (i.e., peak center) not associated with decomposition, respectively. Recited shoulders and onset temperatures are typically have greater uncertanties for their recited temperatures than those recited for the prominent endotherms to which they are associated. A recited shoulder may also become impedded into its associted endotherm and its presence and associated temperature uncertainty is often highly dependent on the temperature scan rate. Embodiments reciting TGA thermal data not associated with decomposition typically have ±2° C. uncertanties for each end of the recited temperature range. Such TGA temperature ranges where a weight loss is recited are typically associated with ±2 wt % uncertanties. For broad or weak enothermic or exothermic DTA, thermal transitions uncertanties double (i.e. ±2° C.) for the inflection point defining those transitions. For prominent DTA endotherms that are usually indicative of melting but are associted with significant TGA wt % losses (i.e., 5-10 wt % or more), such endotherms are typically indicative of decomposition. A transition temperature associated with such an endotherm may be referred to as compound's decomposition temperature and may be associated with an uncertainly of ±5 wt % or more. XRDP data is preferably associated with uncertanties of ±0.10 degress 2-theta. Solid state Raman data is preferably associated with uncertainties of ±1.0 cm⁻¹, more preferably with ±0.5 cm⁻¹. Prominent DTA endotherms not associated with decomposition or prominent exotherms preferably have uncertainties of ±1° C.

1F. Use of a solid state form of androst-5-ene-3α,7β,16α,17β-tetrol (3α-tetrol) in the manufacture of a medicant.

2F. The use according to embodiment 1F wherein the solid state form is crystalline androst-5-ene-3α,7β,16α,17β-tetrol.

3F. The use according to embodiment 1F wherein the solid state form is a crystalline anhydrate of androst-5-ene-3α,7β,16α,17β-tetrol.

4F. The use according to embodiment 3F wherein the crystalline anhydrate is Form Iα or Form IIα 3α-tetrol.

5F. The use according to embodiment 1F or 2F wherein the solid state or crystalline form is Form Iα 3α-tetrol.

6F. The use according to embodiment 1F or 2F wherein the solid state or crystalline form is Form IIα 3α-tetrol.

7F. The use according to embodiment 1F wherein the solid state form is androst-5-ene-3α,7β,16α,17β-tetrol in amorphous form.

8F. Use of a solid state form of androst-5-ene-3α,7β,16α,17β-tetrol in the manufacture of a medicant for the treatment of unwanted inflammation.

9F. The use according to embodiment 8F wherein the unwanted inflammation is a condition or disease associated with chronic, non-production inflammation.

10F. The use according to embodiment 9F wherein the condition or disease is an autoimmune condition or disease.

11F. The use according to embodiment 9F wherein the condition or disease is a metabolic condition or disease.

12F. The use according to embodiment 11F wherein the metabolic condition or disease is type 2 diabetes, obesity, insulin resistance, hyperglycemia, impaired glucose utilization or tolerance, or impaired or reduced insulin synthesis.

13F. The use of any one of embodiments 8F-11F wherein the solid state form is crystalline androst-5-ene-3α,7β,16α,17β-tetrol.

14F. The use of any one of embodiments 8F-11F wherein the solid state form is amorphous androst-5-ene-3α,7β,16α,17β-tetrol.

15F. The use of any one of embodiments 8F-13F wherein the solid state form or crystalline form of androst-5-ene-3α,7β,16α,17β-tetrol is a crystalline anhydrate

16F. The use of any one of embodiments 8F-13F wherein the solid state form or crystalline form of androst-5-ene-3α,7β,16α,17β-tetrol is Form Iα 3α-tetrol.

17F. The use of any one of embodiments 8F-13F wherein the solid state form or crystalline form of androst-5-ene-3α,7β,16α,17β-tetrol is Form IIα 3α-tetrol.

18F. A solid state form of androst-5-ene-3α,7β,16α,17β-tetrol for treating unwanted inflammation.

19F. A solid state form of androst-5-ene-3α,7β,16α,17β-tetrol for treating unwanted inflammation wherein the solid state form is crystalline androst-5-ene-3α,7β,16α,17β-tetrol.

20F. A solid state form of androst-5-ene-3α,7β,16α,17β-tetrol for treating unwanted inflammation wherein the solid state form is androst-5-ene-3α,7β,16α,17β-tetrol in amorphous form.

21F. A solid state form of androst-5-ene-3α,7β,16α,17β-tetrol for treating a metabolic condition wherein the solid state form is crystalline androst-5-ene-3α,7β,16α,17β-tetrol.

32F. A solid state form of androst-5-ene-3α,7β,16α,17β-tetrol for treating a metabolic condition wherein the solid state form is androst-5-ene-3α,7β,16α,17β-tetrol in amorphous form.

33F. A solid state from of androst-5-ene-3α,7β,16α,17β-tetrol for treating a lung inflammation condition or disease wherein the solid state form is crystalline androst-5-ene-3α,7β,16α,17β-tetrol.

34F. A solid state from of androst-5-ene-3α,7β,16α,17β-tetrol for treating a lung inflammation condition or disease wherein the solid state form is androst-5-ene-3α,7β,16α,17β-tetrol in amorphous form.

35F. A solid state form of androst-5-ene-3α,7β,16α,17β-tetrol for treating a liver inflammation condition or disease wherein the solid state form is crystalline androst-5-ene-3α,7β,16α,17β-tetrol.

36F. A solid state form of androst-5-ene-3α,7β,16α,17β-tetrol for treating a liver inflammation condition or disease wherein the solid state form is androst-5-ene-3α,7β,16α,17β-tetrol in amorphous form.

37F. A solid state form of androst-5-ene-3β,7β,16α,17β-tetrol for treating an arthritis condition or disease wherein the solid state form is crystalline androst-5-ene-3α,7β,16α,17β-tetrol.

38F. A solid state form of androst-5-ene-3α,7β,16α,17β-tetrol for treating an arthritis condition or disease wherein the solid state form is androst-5-ene-3α,7β,16α,17β-tetrol in amorphous form.

39F. The solid state form or crystalline form of embodiment 18F, 19F, 21F, 23F, 25F, 27F, 29F, 31F, 33F, 35F or 37F wherein the solid state form or crystalline form of androst-5-ene-3α,7β,16α,17β-tetrol is a crystalline anhydrate.

40F. The solid state form of embodiment 18F, 19F, 21F, 23F, 25F, 27F, 29F, 31F, 33F, 35F or 37F wherein the solid state form or crystalline from of androst-5-ene-3β,7β,16α,17β-tetrol is Form Iα 3α-tetrol.

41F. The solid state form of embodiment 18F, 19F, 21F, 23F, 25F, 27F, 29F, 31, 33F, 35F or 37F wherein the solid state form or crystalline from of androst-5-ene-3β,7β,16α,17β-tetrol is Form IIα 3α-tetrol.

Variations and modifications of these embodiments and other portions of this disclosure will be apparent to the skilled artisan after a reading thereof. Such variations and modifications are within the scope of this invention. The claims in this application or in applications that claim priority from this application will more particularly describe or define the invention. All citations or references cited herein are incorporated herein by reference in their entirety at this location or in additional paragraphs that follow this paragraph.

### EXAMPLES

### General Methods

Raman Spectroscopy—FT-Raman spectra were acquired on a Raman accessory module interfaced to a MAGNA 860™ Fourier transform infrared (FT-IR) spectrometer (Thermo Nicolet). The module uses an excitation wavelength of 1064 nm and an indium gallium arsenide (InGaAs) detector. Approximately 1.5 W of Nd:YVO₄ laser power was used to irradiate the sample. A total of 256 sample scans were collected from 3600-100 cm⁻¹ at a spectral resolution of 4 cm⁻¹ using Happ-Genzel apodization. Wavelength calibration was preformed using sulfur and cyclohexane.

X-ray Powder Diffraction—XRPD patterns were collected using an Intel XRG-3000 diffractometer equipped with a curved position sensitive detector with a 2-theta range of 120° C. An incident beam of Cu Kα radiation (40 kV, 30 mA) was used to collect data in real time at a resolution of 0.03° 2-theta. Prior to the analysis, a silicon standard (NIST SRM 640c) was analyzed to verify the Si 111 peak position. Samples were prepared for analysis by packing them into thin-walled glass capillaries. Each capillary was mounted onto a goniometer head and rotated during data acquisition. The monochromator slit was set at 5 mm by 160 μm, and the samples were analyzed for 5 minutes.

Differential Thermal and Thermogravimetric Analyses—Thermal data was obtained on a Seiko TG/DTA 220U instrument. A 5-8 mg sample of Compound 1 in solid state form was loaded into an aluminum sample pan and tapped down with a glass rod. The sample, in the aluminum sample pan that was uncovered and uncrimped or covered with another pan, was equilibrated at 25° C. and heated under nitrogen purge in at a rate of 10° C./minute, unless otherwise specified, to a final temperature of 300° C.

Abbreviations used: DCM, dichloromethane; DMF, N,N′-dimethyl-formamide; TBDMSCl, t-butyl-dimethylsilyl chloride; TMSCl, trimethylsilyl chloride; ACN, acetonitrile; EtOH, ethanol, MeOH, methanol; EtOAc, ethyl acetate; Et₂O, ethyl ether; THF, tetrahydrofuran; LDA, lithium di-isopropylamide; DHEA, dehydroepiandrosterone; m-CPBA, m-chloroperbenzoic acid.

### Example 1

### Synthesis of androst-5-ene-3α,7β,16α,17β-tetrol (3α-tetrol)

The title compound was prepared according to the following reaction scheme.

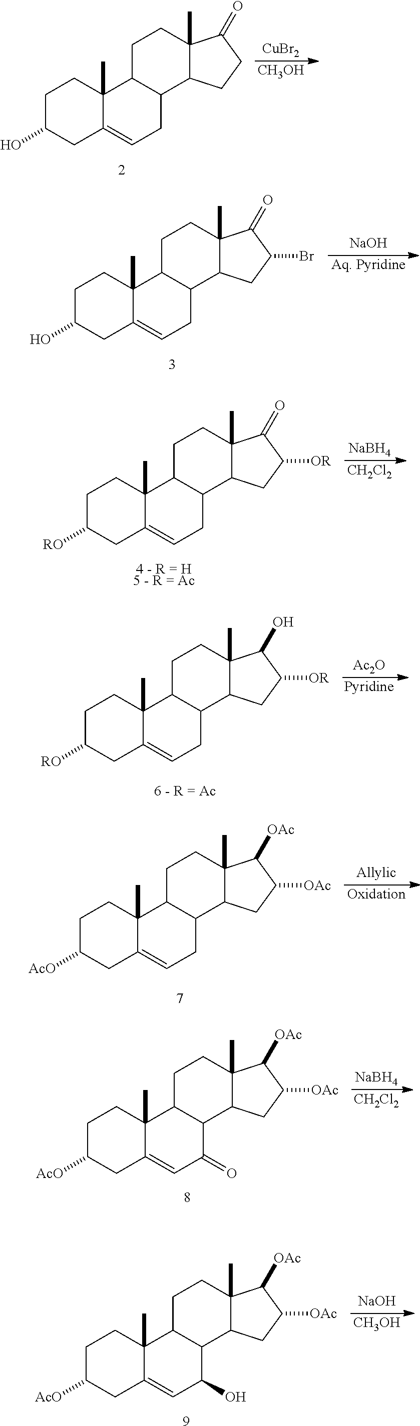

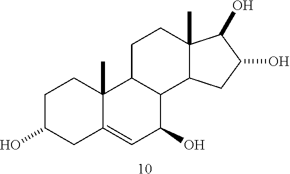

Step 1. 16α-Bromo-5-androst-5-ene-17-one-3α-ol (3): A solution of 5-dehydroandrosterone (2) (17.8 g, 61.7 mmol) in methanol (1.35 L) was refluxed with copper (II) bromide (36.4 g, 163 mmol) with stirring for 19 hours. To the cooled reaction mixture was added water (1.35 L) and dichloromethane (1.5 L). The organic layer was filtered through anhydrous sodium sulfate and the product crystallized as fine needles from methanol (16.7 g, 45.5 mmol, 74%), mp. 195-207° C.

Step 2. 3α,16α-Diacetoxy-androst-5-en-17-one (5): To a solution of 3 (12.0 g, 32.7 mmol) in pyridine (1.032 L) and water (0.247 L) in air was added aqueous 1N sodium hydroxide (90 mL) and the mixture was stirred at room temperature for 15 minutes. The reaction mixture was added to an ice/water mixture containing 1.2 L of 1N hydrochloric acid. After saturating the solution with sodium chloride, it was extracted with ethyl acetate (2×1 L). The combined organic layers were washed with brine (250 mL), filtered through anhydrous sodium sulfate and concentrated. The crude 5-androstene-3α, 16α-diol-17-one (4) was treated with excess acetic anhydride in pyridine at room temperature overnight and purified by column to give 5 (7.46 g, 19.2 mmol, 59%) as prisms from methanol, mp. 172.7-173.7° C.

Step 3. 3α,16α,17β-Tri-acetoxy-androst-5-ene (7): To a solution of enediolone diacetate 5 (7.46 g, 19.2 mmol) in dichloromethane (45 mL) and methanol (120 mL) at 0° C. was added sodium borohydride (950 mg). The solution was stirred at 0° C. for 1 hour. After addition of excess acetic acid the reaction mixture was partitioned between dichloromethane and water. The organic layer was filtered through anhydrous sodium sulfate and concentrated to yield a mixture of the 17a (minor) and 17p (major) epimers. This mixture was purified by flash chromatography (25% ethyl acetate in hexanes) to give 6.1 g (15.6 mmol, 81%) of the 17β epimer 6. Mp 126.9-128.6° C. The triacetate was made from 6 by treatment with excess acetic anhydride in pyridine at room temperature overnight. Purification by column chromatography gave 6.0 g 7 (13.9 mmol, 89%).

Step 4. 3α,16α,17β-Tri-acetoxy-androst-5-en-7-one (8): A solution of the triacetate 7 (6.0 g, 13.9 mmol) in benzene (255 mL) was treated with celite (25.5 g), pyridinium dichromate (31.5 g) and 70% tert-butyl hydrogen peroxide (9.0 mL) and stirred at room temperature for 19 hours. Anhydrous diethyl ether (255 mL) was added and reaction mixture was cooled in an ice bath for 1 hour. The resulting solid was filtered off and washed with ether (2×50 mL). The combined organic portions were concentrated and purified by flash chromatography (29% ethyl acetate in hexanes) to give 3.45 g of 8 (7.7 mmol, 55%).

Step 5: Androst-5-ene-3α,7β,16α,17β-tetrol (3α-tetrol). To a solution of 8 (3.45 g, 7.7 mmol) in dichloromethane (15 mL) and methanol (30 mL) at 0° C. was added sodium borohydride (1.0 g) and the solution was stirred at 0° C. for 2 hours. After addition of excess acetic acid (1.5 mL) the reaction mixture was partitioned between dichloromethane and water. The organic layer was filtered through anhydrous sodium sulfate and concentrated to yield a mixture of the 7α (minor) epimer (9a) and 7β (major) epimer (9b). This mixture was saponified in methanol (100 mL) with 1N sodium hydroxide (60 mL) overnight at room temperature. The crude tetrols were recovered by partitioning the saponification mixture between ethyl acetate and brine. The epimers were separated by HPLC to give 3α-tetrol as the major product. Selected ¹H-NMR peaks (CD₃OD, ppm): 5.23 (s, 1H), 4.01 (m, 2H), 3.80 (m, 1H), 3.38 (d, 1), 2.53 (d, 1H), 2.10 (d, 1H), 2.08 (d, 1H), 1.0-1.9 (m, 15H), 1.04 (s, 3H), 0.77 (s, 3H).

### Example 2

Alternative synthesis of androst-5-ene-3α,7β,16α,17β-tetrol (3α-tetrol): The title compound was alternatively prepared according to the following reaction scheme.

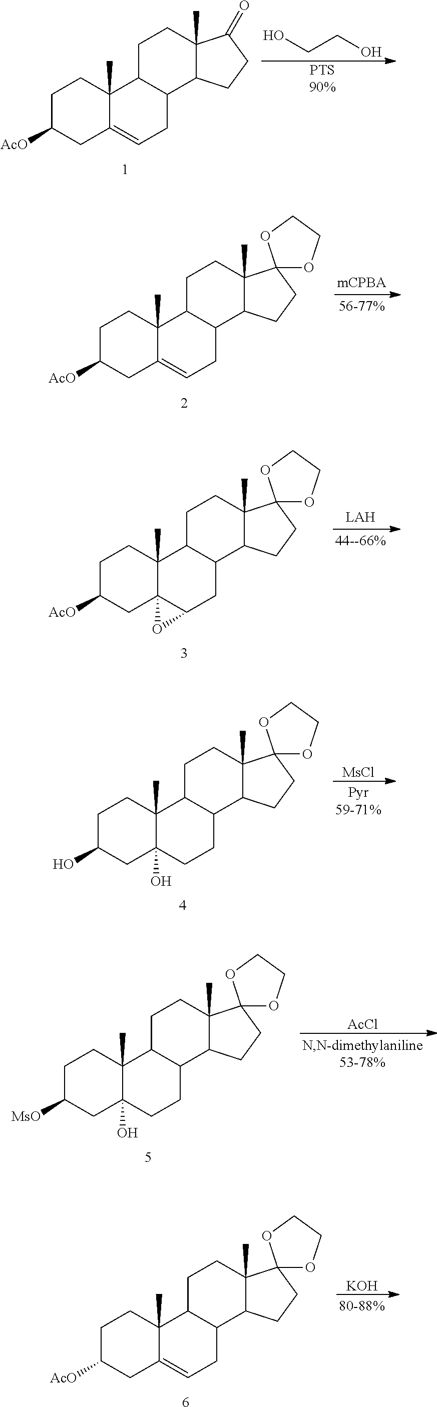

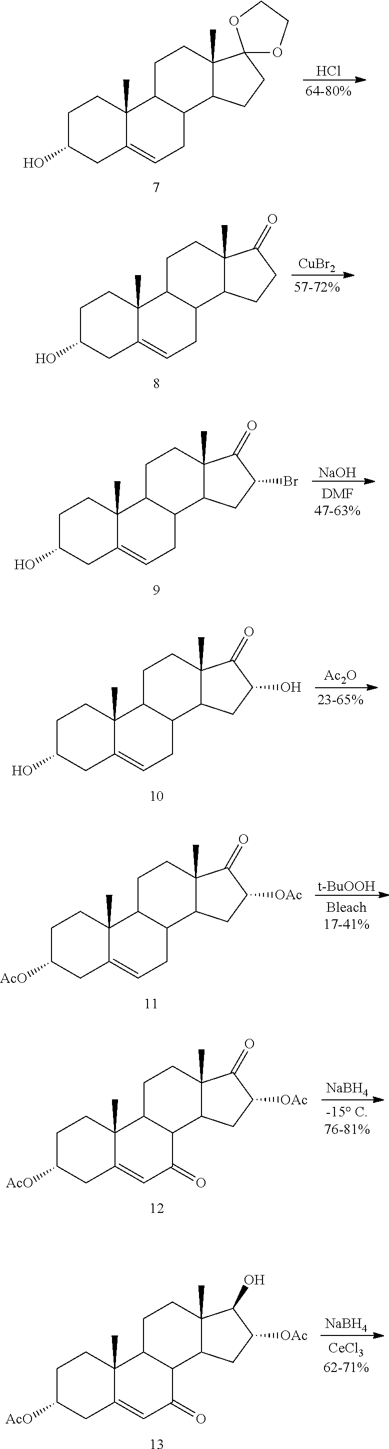

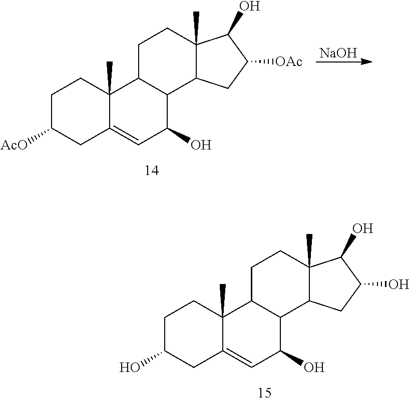

Step 1. 17,17-Ethylenedioxy-3β-acetoxy-androst-5-ene (2): A solution of 500 g 3β-acetoxy-dehydroepiandrosterone (1), triethylorthoformate (900 mL), ethylene glycol (315 mL) and p-toluenesulfonic acid (40 g) was heated under nitrogen and refluxed for 3 hours. The solution was then cooled to 60° C. and anhydrous ethanol (400 mL) and pyridine (10 mL) was added. The mixture was then cooled and allowed to stand in freezer for 16 hours. The resulting solid was collected by vacuum filtration, washed with 50% ethanol solution (2 L) and dried at 50° C. under vacuum for 16 hours to yield 510 g of 2, (90% yield).

Step 2. 17,17-Ethylenedioxy-5α,6α-epoxy-3β-acetoxy-androstane (3): In a stirred solution of 2 (500 g) in DCM (2.68 L) cooled to −5° C. was added a solution of m-CPBA (295 g) in DCM (4 L) dropwise during which the temperature of the reaction mixture was kept at ≧−2° C. The mixture was allowed to warm to room temperature after the addition was complete and stirred at room temperature for 2 additional hours. Saturated Na₂SO₃ solution (1.25 L) was added and the resulting mixture was stirred for 30 min. DCM (5 L) and water (2.5 L) were then added so that all of the solids were dissolved. The organic layer was separated and washed with saturated NaHCO₃ solution (3×1.5 L) and brine (2.5 L), dried over MgSO₄, and concentrated to provide 560 g crude 3. The crude was recrystallized in methanol 5 times to remove the β-epoxide isomer and provide 343 g purified 5α,6α-epoxide 3 (66% yield).

Step 3. 17,17-Ethylenedioxy-androstane-3β,5α-diol (4): To a mixture of LiAlH₄ (64 g) in dry THF (1.5 L) cooled with a NaCl/ice bath was added a solution of 3 (343 g) in dry THF (2 L) dropwise so as to maintain the internal reaction temperature ≧35° C. After the addition was complete, the mixture was heated to reflux for 3 hours. The mixture was then cooled with a NaCl/ice bath and excess LiAlH₄ was quenched with ethyl acetate (350 mL). 12.5 NaOH solution (250 mL) was added and the mixture was filtered. The collected solids were washed with THF and the combined organic filtrates were dried over MgSO₄ and concentrated to provide 360 g crude 4. The crude was recrystallized in methanol 4 times to remove the 3,6-diol isomer to provide 206 g purified 3β,5α-diol 4 (67% yield).

Step 4. 17,17-Ethylenedioxy-3β-methanesulfonyloxy-androstane-5α-ol: To a stirred solution of 4 (206 g) in dry DCM (1.8 L) was added pyridine (380 g). The solution was then cooled with ice water bath whereupon methanesulfonyl chloride (240 mL) was added dropwise so that the internal reaction temperature was kept at ≧10° C. After addition, the reaction mixture was stirred for 16 hours at room temperature then washed with water (1.5 L), 5% H₂SO₄ (3×2 L), 5% NaOH (3×2 L) and water (3×2 L), dried over MgSO₄, and concentrated to remove solvent to provide 179 g crude 5 (71% yield).

Step 5. 17,17-Ethylenedioxy-3α-acetoxy-androst-5-ene (6): To a solution of 5 (179 g) in chloroform (2 L) was added N,N′-dimethylaniline (670 mL) and acetyl chloride (670 mL). The mixture was then heated to reflux for 5 hours. After removal of solvent under vacuum, ethyl acetate (2 L) was added to the residue. The resulting solution was washed with water (2×2 L), 5% H₂SO₄ (3×2 L), water (3×2 L), 5% NaOH (3×2 L), and water (3×2 L) and dried over MgSO₄. The residue obtained after concentration in vacuo was recrystallized in methanol to provide 113 g purified 6 (72% yield).

Step 6. 17,17-Ethylenedioxy-androst-5-ene-3α-ol (7): To a solution of 6 (113 g) in methanol (1.5 L) was added a solution of KOH (83 g) in water (1.5 L). The resulting mixture was refluxed for 1 hour, then cooled to room temperature. A solid precipitated that was collected by vacuum filtration, washed with water (150 mL), and dried to yield 72 g crude 7 (81% yield) that was used in the next step without further purification.

Step 7. Dehydroandrosterone (8): To a stirred solution of 7 (72 g) in ethanol (500 mL) was added 1N HCl (500 mL). The solution was refluxed for 5 hours, then cooled to room temperature to give a white solid that was collected by vacuum, washed with water (100 mL) and dried to provide 49 g of 8 (78% yield).

Step 8. 16α-Bromo-androst-5-en-17-one-3α-ol (9): To a solution of 8 (49 g) in methanol (80 ml) was added CuBr₂ (83 g). The mixture was refluxed for 3 hours then cooled to room temperature and filtered. The collected solids were dissolved in THF (150 mL) and filtered. Concentration of this filtrate provided 44 g crude 9 (71% yield) that was used in the next step without further purification.

Step 9. Androst-5-en-17-one-3α,16α-diol (10): To a solution of 9 (44 g) in DMF (600 mL) cooled to 0° C. was added 1N NaOH (131 mL) dropwise. The mixture was stirred for 30 min at room temperature after addition was complete, followed by adjusting the pH of the solution to 7.0 with 1N HCl. The solution was then poured into water (750 mL) to give a white precipitate that was collected by vacuum filtration. The collected solids were washed with water to give 35 g crude product. The crude was recrystallized in 1:1 methanol/water (200 mL) to provide 22 g purified 10 (62% yield).

Step 10. 3α,16α-Diacetoxy-androst-5-en-17-one (11): To a solution of 10 (22 g) in DCM (150 mL) was added pyridine (17.1 g) and acetic anhydride (18.3 g). The reaction mixture was stirred at room temperature for 16 hours, then washed with water, 1N HCl, water and saturated NaHCO₃, and dried over Na₂SO₄. After filtration, the filtrate was concentrated to give 21 g crude product. The crude was purified by flash chromatography using hexane/EtOAc to yield 18 g 11 (65% yield).

Step 11. 3α,16α-Diacetoxy-androst-5-en-7,17-dione (12): To a solution of 11 (18 g) in ethyl acetate (150 mL) was added t-butyl hydroperoxide (36 g) followed by 8% NaOCl solution (72 g) added dropwise over 10 hours. The aqueous layer of the reaction mixture was separated and extracted with ethyl acetate (2×100 mL). The combined organic layers were washed with 10% NaHSO3 (2×100 mL) and brine (3×100 mL), dried over Na₂SO₄, and concentrated to give crude product (20 g). The crude was purified by flash chromatography using hexane/EtOAc to yield 7.6 g of 12 (41% yield).

Step 12. 3α,16α-Diacetoxy-androst-5-en-7-one-17β-ol (13): To a solution of 12 (7.6 g) in methanol (50 mL) and THF (50 mL) cooled to −15° C. was added NaBH₄ (0.50 g) in aliquots over 15 min. The solution was stirred for 30 more minutes at −15° C. then poured into ice water and extracted with EtOAc (3×100 mL). The combined organic extracts were washed with brine (100 mL), dried over Na₂SO₄, and concentrated to give 6.0 g crude 13 (79% yield), which was used in the next step without further purification.

Step 13. 3α,16α-Diacetoxy-androst-5-ene-7β,17β-diol: To a solution of 13 (6.0 g) in methanol (50 mL) and THF (50 mL) cooled to 0° C. was added CeCl_{3.7}H₂O (6.6 g). The mixture was stirred until the solids dissolved whereupon NaBH₄ (0.68 g) was added in aliquots over 15 min. The solution was stirred for 30 more minutes at 0° C., then poured into ice water and extracted with EtOAc (3×100 mL). The combined organic extracts were washed with brine (100 mL), dried over Na₂SO₄, and concentrated to give 5.8 g crude product. The crude was further purified by flash chromatography using hexane/EtOAc to yield 3.72 g purified 14, (62% yield).

Step 14: Androst-5-ene-3α,7β,16α,17β-tetrol (3α-tetrol): To a solution of 14 (4.0 g) in MeOH (150 mL) at room temperature was added NaOH (2.2 g) in water (20 mL) and the solution stirred overnight. The solution was then cooled to 0 C and 1N HCl (28 mL) added to produce a neutral (pH=7) solution. The solution was then concentrated in vacuo to remove most of the methanol and then water (150 mL) added and the solution frozen. The water was removed by use of a lyophilizer and a powder collected. This was then stirred with THF (50 mL) for 30 mins at room temperature and filtered to remove the THF. The powder was dissolved in methanol (5 mL) and diethyl ether added dropwise to precipitate out a solid material. This product was then filtered and dried in vacuo to produce 3.1 g of 15 (98% yield).

### Example 3

Crystalline Form Iα 3α-tetrol: To 50 mg of 3α-tetrol in 0.2 mL MeOH at room temperature was added 0.2 mL acetone to crash precipitate crystalline material. Collection by vacuum filtration provided 39 mg Form Iα.

TABLE 1
Observed XRPD peaks for Form Iα 3α-tetrol

| degree 2θ | d space (Å) | Intensity (%) |
|---|---|---|
| 7.6 ± 0.1 | 11.602 ± 0.154 | 56 |
| 8.4 ± 0.1 | 10.526 ± 0.127 | 3 |
| 10.7 ± 0.1 | 8.238 ± 0.077 | 4 |
| 13.7 ± 0.1 | 6.445 ± 0.047 | 37 |
| 15.3 ± 0.1 | 5.791 ± 0.038 | 35 |
| 16.1 ± 0.1 | 5.502 ± 0.034 | 100 |
| 16.5 ± 0.1 | 5.382 ± 0.033 | 26 |
| 17.0 ± 0.1 | 5.213 ± 0.031 | 25 |
| 17.8 ± 0.1 | 4.994 ± 0.028 | 41 |
| 19.8 ± 0.1 | 4.491 ± 0.023 | 21 |
| 20.9 ± 0.1 | 4.248 ± 0.020 | 10 |
| 21.1 ± 0.1 | 4.213 ± 0.020 | 9 |
| 21.5 ± 0.1 | 4.131 ± 0.019 | 6 |
| 22.2 ± 0.1 | 4.010 ± 0.018 | 28 |
| 23.1 ± 0.1 | 3.855 ± 0.017 | 3 |
| 23.8 ± 0.1 | 3.731 ± 0.015 | 8 |
| 25.1 ± 0.1 | 3.551 ± 0.014 | 5 |
| 25.9 ± 0.1 | 3.445 ± 0.013 | 6 |
| 27.2 ± 0.1 | 3.277 ± 0.012 | 8 |
| 28.4 ± 0.1 | 3.145 ± 0.011 | 12 |
| 29.3 ± 0.1 | 3.044 ± 0.010 | 4 |

The DTA thermogram of Form Iα, obtained with the sample uncovered, exhibits an exotherm centered at about 154° C. followed by a prominent endotherm centered at about 225° C. (onset at about 216° C.) that is associated with negligible weight loss in the thermogravimetric thermogram from between about 60° C. to about 280° C., with a temperature scan rate of 10° C./min. These temperature transitions are consistent with Form Iα existing as an anhydrate that undergoes conversion to a more stable polymorphic form before finally melting.

### Example 4

Crystalline Form IIα 3α-tetrol: To 50 mg of 3α-tetrol prepared according to example 4 in 0.2 mL denatured EtOH at room temperature was added 0.2 mL Et₂O. Afterwards, the mixture was allowed to stand at between about −10 to −20° C. in a freezer for 10 days. The resulting crystalline material was collected by vacuum filtration provide 8 mg of Form IIα.

The DTA thermogram of Form IIα, obtained with the sample uncovered, exhibits a prominent endotherm centered at about 243° C. An onset temperature at about 230° C. appears to overlap with a shoulder to the prominent endotherm that may represent a transition to a more stable polymorphic form. Negligible weight loss in the thermogravimetric thermogram from between about 60° C. to about 280° C., with a temperature scan rate of 10° C./min is observed. This thermal data is consistent with Form IIα existing as an anhydrate that may undergo partial melting before conversion to a more stable polymorphic form which then finally melts.

### Example 5

Amorphous 3α-tetrol: To a solution of 5 g 3α,16α,17β-triacetoxy-7β-androst-5-ene-7β-ol (9b, example 1) in 200 mL MeOH at room temperature was added 2.7 g of NaOH in 20 mL of water. After stirring overnight. the solution was cooled to 0° C. whereupon 1N HCl was added to pH=7 (about 22 mL). The solution was concentrated in vacuo to a slurry and lyophilized. The resulting powder was dissolved in 100 mL THF and filtered to remove insoluble material. To the filtrate was added 5 L of MeOH and 100 mL of ether to precipitate out a solid out. Filtration provided 1.3 g of solid material. The filtrate was washed with brine and concentrated in vacuo to give another 1.9 grams of solid material. The combined solids were dissolved in 5 mL MeOH, and 150 mL of diethyl ether was added dropwise over a period of 5 mins. After stirring for 1 h, the resulting solids were collected by vacuum filtration and dried in vacuo to provide 2.55 g of amorphous 3α-tetrol.

TABLE 2
Peak listing for absorptions for Raman
spectrum of amorphous 3α-tetrol

| cm⁻¹ | Intensity |
|---|---|
| 229 | 2.04 |
| 299 | 0.99 |
| 316 | 1.27 |
| 337 | 1.48 |
| 349 | 1.5 |
| 372 | 1.19 |
| 380 | 1.16 |
| 426 | 1.23 |
| 443 | 1.2 |
| 453 | 1.05 |
| 480 | 1.9 |
| 513 | 0.9 |
| 530 | 0.65 |
| 550 | 1.27 |
| 565 | 1.08 |
| 604 | 0.62 |
| 623 | 0.71 |
| 656 | 1.62 |
| 686 | 0.59 |
| 719 | 0.72 |
| 731 | 0.88 |
| 775 | 1.8 |
| 837 | 1.44 |
| 852 | 1.25 |
| 893 | 1.54 |
| 910 | 1.36 |
| 931 | 0.78 |
| 943 | 0.79 |
| 962 | 0.94 |
| 999 | 1.38 |
| 1036 | 1.1 |
| 1063 | 1.25 |
| 1109 | 1.02 |
| 1130 | 1.64 |
| 1151 | 1.82 |
| 1201 | 1.36 |
| 1238 | 1.67 |
| 1269 | 1.34 |
| 1306 | 1.3 |
| 1333 | 1.66 |
| 1381 | 1.05 |
| 1442 | 2.93 |
| 1454 | 2.47 |
| 1626 | 0.86 |
| 1674 | 2.61 |
| 2858 | 4.99 |
| 2895 | 6.5 |
| 2939 | 9.25 |
| 2968 | 5.79 |

XRPD for amorphous 3α-tetrol exhibits a broad, featureless band from about 11 degree 2-theta to about 20 degree 2-theta centered between about 16 to 17 degree 2-theta. Such a halo is indicative of amorphous material. The DTA thermogram of amorphous 3α-tetrol, obtained with the sample covered and a temperature ramp of 10° C./min, exhibits a prominent exotherm at 166.0° C. and a endotherm at about 225.2° C. with a shoulder at about 220° C. The prominent exotherm of 166.0° C. is indicative of amorphous 3α-tetrol transitioning to crystalline material, which finally melts at 225.2° C. The TG thermogram exhibits considerable and abrupt weight loss (23%) at about 140° C., which is most likely due to loss of non-specifically absorbed solvent.

### Example 6

Treatment of inflammation—Lung inflammation conditions: The compound androstane-3α,16α,17α-triol was found to have biological properties that make the compound superior as an agent to treat an inflammation condition, including lung inflammation conditions such as asthma. Specifically, the use of the compound was not accompanied by a rebound in IL-13, which is a known side effect of anti-inflammatory glucocorticoid compounds such as dexamethasone. The IL-13 rebound after glucocorticoid makes an asthma patient more prone to have subsequent acute flare, so an anti-inflammatory agent that does not do this would be advantageous. This lack of an IL-13 rebound was unexpected.

The capacity of androstane-3α,16α,17α-triol to limit eosinophil burden and to reduce key inflammatory mediators (IL-5, IL-13, cysteinyl leukotrienes) was observed in the ovalbumin (OVA) sensitized mouse model of asthma. BALB/c mice were sensitized by intraperitoneal injection with OVA (in alum adjuvant) on days 1, and 12. Airways were challenged with OVA on days 28 and 30 by delivery of OVA to the lung, or with saline. On day 31, six mice were with saline and 6 mice challenged with OVA were sacrificed and lung tissue was analyzed. The remaining animals were divided into 6 groups (6 mice per group). Groups of the mice were treated once daily by subcutaneous injection as follows. Group 1 vehicle control (0.1% carboxymethyl cellulose, 0.9% saline, 2% Tween 80, 0.05% phenol). Group 2 dexamethasone (5 mg/kg). Group 3 androstane-3α,16α,17α-triol (1 mg/mouse). Three animals in groups 1-3 were sacrificed on day 35 at 1 hr after final treatment and the remaining 3 animals in groups 1-3 were sacrificed on day 38.

As shown in the table below, the androstane-3α,16α,17α-triol did not generate an IL-13 increase that was observed with animals that had been treated with dexamethasone.

| | IL-13 |
|---|---|
| Treatment | (pg/mL) |
| saline control | 220 |
| ovalbumin | 230 |
| vehicle (day 35) | 220 |
| dexamethasone (day 35) | 340 |
| androstane-3α,16α,17α-triol (day 35) | 195 |
| vehicle (day 38) | 190 |
| dexamethasone (day 38) | 390 |
| androstane-3α,16α,17α-triol (day 38) | 210 |

In addition to a reduction in the day 38 IL-13 rebound after challenge, the animals treated with 3α,16α,17α-trihydroxyandrostane had a reduced level of IL-5 in lung tissue (90 pg/mL) compared to the dexamethasone treated group (145 pg/mL). The IL-5 level in the vehicle control group was 75 pg/mL at day 38. Other formula 1 compounds described herein were used in this manner to identify their capacity to treat or ameliorate inflammation without an IL-13 and/or IL-5 rebound effect, including trihydroxyandrostane-3β,16β,17β-triol, androstane-3β,16α,17α-triol, androstane-3β,16β,17α-triol, androst-5-ene-2α,3β,16α,17β-tetrol, androst-5-ene-3β,7β,16α,17β-tetrol and 17α-ethynylandrost-5-ene-3β,7β,17β-triol. These results show that steroid triols and tetrols can be used to treat a lung inflammation condition such as asthma in vivo.

In another protocol, a population of mast cells was cultivated from murine bone marrow as follows. Briefly, bone marrows from Balb/C mice were flushed from the femur using PBS and a 27 g needle. The cells were cultured in a mixture of ⅔ RPMI-1640 with 19% FBS and cells that secreted IL-3. The bone marrow cells were allowed to differentiate for 18-25 days in the IL-3-containing mixture before being used for experiments. Bone marrow cells cultured in this manner have a phenotype similar to mucosal mast cells and are referred to as bone marrow-derived mast cells (BMMC).

The homogeneity of the in vitro propagated mast cells was checked by conventional flow cytometric techniques and staining for cell-type specific markers. Between days 14 and 21 of propagation, mature mast cells were harvested and prepared for the test cultures. The objective was to assess of the effect of compounds such as dehydroepiandrosterone on mast cell stimulus-coupled degranulation. Prepared mast cells were dispensed into test culture wells at a density of 1×10⁷ cells/mL. In control cultures, mast cells were induced to degranulate after cross linking of IgE receptors with IgE antigen-antibody complexes. In parallel groups of cultures mast cells were preincubated dehydroepiandrosterone at various doses followed by activation using anti-IgE antibody. There was no detectable degranulation of mast cells as measured by release of β-glucuronidase from cytosolic storage granules of the cells in the absence of the stimulus. Introduction of anti-Ig-E receptor antibody to the cultures caused a significant release of β-glucuronidase. When mast cells were exposed to dehydroepiandrosterone alone, there was no measurable degranulation. However, mast cells pre-exposed to doses of 100 μM dehydroepiandrosterone for 5 to 10 minutes before activation with anti-IgE antigen-antibody complexes, exhibited approximately 70% inhibition of degranulation. Lower levels of dehydroepiandrosterone showed proportionately less capacity to inhibit degranulation. In similar protocols, steroid triols or tetrols such as 17α-ethynylandrost-5-ene-3β,7β,17β-triol, androst-5-ene-3β,7β,16α,17β-tetrol or androst-5-ene-3α,7β,16α,17β-tetrol were 10-1000 fold more potent than dehydroepiandrosterone.

What is claimed is:

1. Crystalline androst-5-ene-3α,7β,16α,17β-tetrol.

2. The crystalline androst-5-ene-3α,7β,16α,17β-tetrol of claim 1 wherein crystalline androst-5-ene-3α,7β,16α,17β-tetrol is a crystalline anhydrate.

3. The crystalline androst-5-ene-3α,7β,16α,17β-tetrol of claim 1 wherein crystalline androst-5-ene-3α,7β,16α,17β-tetrol is Form Iα or Form IIα 3α-tetrol.

4. The crystalline androst-5-ene-3α,7β,16α,17β-tetrol of claim 3 wherein crystalline androst-5-ene-3α,7β,16α,17β-tetrol is Form Iα 3α-tetrol characterized by (1) an XRPD pattern having three or more peaks selected from the group consisting of about 7.6, 16.1, 17.8, 19.8 and 22.2 degree 2-theta or (2) DTA thermogram, obtained with a temperature ramp of 10° C./min, having a prominent endotherm centered at about 224° C. and an exotherm centered at about 154° C. or (1) and (2).

5. The crystalline androst-5-ene-3α,7β,16α,17β-tetrol of claim 4 wherein the XRPD pattern further has one or more peaks selected from the group consisting of about 13.7, 15.3, 16.5, 17.0 and 20.9 degree 2-theta.

6. The crystalline androst-5-ene-3α,7β,16α,17β-tetrol of claim 4 wherein the 224° C. DTA endotherm has an onset temperature of about 216° C.

7. The crystalline androst-5-ene-3α,7β,16α,17β-tetrol of claim 4 wherein crystalline androst-5-ene-3α,7β,16α,17β-tetrol is further characterized by TGA thermogram, obtained with a temperature ramp of 10° C./min, having (1) negligible weight loss from about 60° C. to about 140° C. or (2) about 2% wt loss from about 60° C. to about the onset of the prominent endotherm or (1) and (2).

8. The crystalline androst-5-ene-3α,7β,16α,17β-tetrol of claim 3 wherein crystalline androst-5-ene-3α,7β,16α,17β-tetrol is Form IIα 3α-tetrol characterized by (1) DTA thermogram, obtained with a temperature ramp of 10° C./min, having a prominent endotherm centered at about 243° C.

9. The crystalline androst-5-ene-3α,7β,16α,17β-tetrol of claim 8 wherein the 243 oC DTA endotherm has (1) an onset temperature of about 229° C. or (2) a shoulder between about 230 to 240° C. or (1) and (2).

10. Amorphous androst-5-ene-3α,7β,16α,17β-tetrol.

11. The amorphous androst-5-ene-3α,7β,16α,17β-tetrol of claim 10 characterized by (1) XRPD pattern having a broad band from about 11 degree 2-theta to about 20 degree 2-theta or a broad band centered between about 16 to 17 degree 2-theta or (2) DTA thermogram, obtained with a temperature ramp of 10° C./min, having a prominent exotherm centered at about 166° C. or (1) and (2).

12. The amorphous androst-5-ene-3α,7β,16α,17β-tetrol of claim 11 wherein the DTA thermogram further has an endotherm centered at about 225° C.

13. The amorphous androst-5-ene-3α,7β,16α,17β-tetrol of claim 12 wherein the 225° C. DTA endotherm has a shoulder at about 220° C.

14. The amorphous androst-5-ene-3α,7β,16α,17β-tetrol of claim 11 further characterized by TGA thermogram with negligible % weight loss between about 60° C. to about 140° C.

15. A composition comprising one or more excipients and a solid state form of androst-5-ene-3α,7β,16α,17β-tetrol.

16. The composition of claim 15 wherein the solid state form is crystalline androst-5-ene-3α,7β,16α,17β-tetrol.

17. The composition of claim 15 wherein the solid state form is a crystalline anhydrate.

18. The composition of claim 17 wherein the crystalline anhydrate is Form Iα 3α-tetrol.

19. The composition of claim 17 wherein the crystalline anhydrate is Form IIα 3α-tetrol.

20. A method of preparing a liquid formulation comprising admixing a solid state form of androst-5-ene-3α,7β,16α,17β-tetrol with a liquid excipient.

21. The method of claim 20 wherein the solid state form is crystalline androst-5-ene-3α,7β,16α,17β-tetrol.

22. The method of claim 20 the solid state form of androst-5-ene-3α,7β,16α,17β-tetrol is a crystalline anhydrate.

23. The method of claim 13 wherein the crystalline anhydrate is Form Iα or Form IIα 3α-tetrol.

24. The method of claim 20 wherein the solid state form is amorphous 3α-tetrol.

25. A method of treating unwanted inflammation, comprising administering an effective amount of a solid formulation to a subject in need thereof wherein the solid formulation comprises a solid state form of androst-5-ene-3α,7β,16α,17β-tetrol and one or more excipients.

26. The method of claim 25 wherein the solid state form is crystalline androst-5-ene-3α,7β,16α,17β-tetrol.

27. The method of claim 25 wherein the solid state form is a crystalline anhydrate.

28. The method of claim 27 wherein the crystalline anhydrate is Form Iα or Form IIα 3α-tetrol.

29. The method of claim 25 wherein the solid state form is amorphous 3α-tetrol.

30. The method of claim 25 wherein the unwanted inflammation is a condition or disease associated with chronic, non-production inflammation.

31. The method of claim 30 wherein the condition or disease is an autoimmune condition or disease.

32. The method of claim 30 wherein the condition or disease is a metabolic condition or disease.

33. The method of claim 31 wherein the autoimmune disease is Type 1 diabetes.

34. The method of claim 31 wherein the autoimmune disease is a lupus condition, systemic lupus erythematosus or discoid lupus

35. The method of claim 30 wherein the condition or disease is an arthritis condition or rheumatoid arthritis.

36. The method of claim 30 wherein the condition or disease is an inflammatory bowel disease, ulcerative colitis or Crohn's disease (regional enteritis).

37. The method of claim 30 wherein the condition or disease is a lung inflammation condition, cystic fibrosis, chronic obstructive pulmonary disease (COPD), acute respiratory distress syndrome, acute asthma, chronic asthma, emphysema, acute bronchitis, allergic bronchitis, chronic bronchitis, a fibrosing alveolitis (lung fibrosis) condition or subepithelial fibrosis in patients having chronic bronchitis, asthma and/or COPD.

38. The method of claim 30 wherein the condition or disease is a neurodegenerative condition, Parkinson's disease or Alzheimer's disease.

39. The method of claim 30 wherein the condition or disease is a hyperproliferation condition or cancer.

40. The method of claim 30 wherein the condition or disease is a liver cirrhosis condition, nonalcoholic steatohepatitis (NASH) or nonalcoholic fatty liver disease.

41. The method of claim 32 wherein the metabolic condition or disease is type 2 diabetes, obesity, insulin resistance, hyperglycemia, impaired glucose utilization or tolerance, or impaired or reduced insulin synthesis.
